(19) United States
(12) Patent Application Publication
(10) Pub. No.: US 2009/0254008 A1
(43) Pub. Date: Oct. 8, 2009

(54) SYSTEMS, DEVICES, AND METHODS TO CONCURRENTLY DELIVER ULTRASOUND WAVES HAVING THERMAL AND NON-THERMAL EFFECTS

(76) Inventor: Donald J. Shields, JR., Arcadia, CA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 12/362,425

(22) Filed: Jan. 29, 2009

Related U.S. Application Data
(60) Provisional application No. 61/024,506, filed on Jan. 29, 2008.

(51) Int. Cl. A61N 7/02

(52) U.S. Cl. 601/3

(57) ABSTRACT

Systems, devices, and methods for delivering ultrasonic treatment to a subject. An ultrasound therapy device includes one or more waveform generators, one or more transducers, one or more sensors, and at least one controller. In some embodiments, the one or more waveform generators are configured to generate a first drive signal and at least a second signal, the first or second signals having at least a first waveform segment and a second waveform segment different from the first waveform segment. In some embodiments, the systems, devices, and methods are operable to provide thermal and non-thermal ultrasonic waveforms.

### CROSS-REFERENCE TO RELATED APPLICATION

This application claims the benefit under 35 U.S.C. § 119(e) of U.S. Provisional Patent Application No. 61/024,506 filed Jan. 29, 2008, where this provisional application is incorporated herein by reference in its entirety.

### BACKGROUND

1. Field of Technology

This disclosure generally relates to the field of therapeutic ultrasound and, more particularly, to systems, devices, and methods for providing ultrasound therapy to a subject.

2. Description of the Related Art

Humans and animals are composed of cells organized into various functional units or tissues, for example, bone, muscle, tendon, ligament, and cartilage. These and other commonly injured tissues are sometimes treated with therapeutic ultrasound.

Often therapeutic ultrasound devices output a single, fixed, and non-varying waveform. It is usually necessary to interrupt therapy or reprogram the ultrasound device before initiating a new therapeutic output or delivering a different waveform. Currently, applicants know of no apparatuses or methods that deliver concurrent therapeutic low intensity, non-thermal waveforms and moderate intensity, thermally active waveforms.

Ultrasound therapy often employs transducers to deliver ultrasound energy to the injured tissues. The thermal effects commonly associated with this type of therapy can, however, damage the target tissue if the transducers are not kept in constant motion. Because of the risk of damaging target tissue, conventional ultrasound devices necessitate the use of trained and knowledgeable operators.

Ultrasound treatment is an attended therapy that requires a clinician to be present to move the ultrasound head over the treatment area. Often the clinician places a small layer of gel between the transducer and the tissue, and utilizes a moving transducer technique while applying the ultrasonic therapy. Transducer movement is generally necessary to treat areas larger than the area of the transducer, or to avoid damage caused by signal “hot spots”. The movement of the transducer over the gel covering the treatment area causes the gel to be displaced and often requires constant attended application of the gel.

The speed and/or rate that the transducer moves during treatment varies widely from one clinician to another. Moreover, moving the transducer too fast, not using enough coupling medium, not moving the transducer, trying to treat too large of an area, not keeping the transducer in contact with the patient, and other faults often results in misuse of the ultrasound device. Since treatments must be supervised by a clinician, the patient is often limited to specific treatment times necessitating multiple treatments to complete the therapy.

The effects of therapeutic ultrasound on living tissues vary. For example, ultrasound typically has a greater affect on highly organized, structurally rigid tissues such as bone, tendons, ligaments, cartilage, and muscle. Due to their different depths within the body, however, the different tissue types require different ultrasonic frequencies for effective treatment. In addition, tissues respond to ultrasound in different ways depending on the chronicity of the injury to the tissue. Accordingly, acute and chronic injuries are treated differently.

Utilizing these scientific principles the above-mentioned apparatuses and methods use ultrasonic energy for in vivo therapeutic treatment of bone tissue with carrier frequencies and therapeutic ultrasound pulses. Typical apparatuses often allow for the selection of certain treatment parameters such as ultrasound frequency, pulse intensity, etc., but typically produce a single, fixed waveform during each treatment application. Moreover, the typical ultrasound apparatuses are designed to treat a single type of tissue with a single, specific and fixed ultrasonic frequency, pulse intensity, pulse ratio, pulse duration, and pulse repetition rate during each therapeutic use. Because these apparatuses generally employ singular waveforms, the transducer treatment elements usually require substantial movement about a treatment area to avoid thermally damaging the target tissue.

The present disclosure is directed to overcome one or more of the shortcomings set forth above, and provide further related advantages.

### BRIEF SUMMARY

In one aspect, the present disclosure is directed to an ultrasound therapy device for delivering ultrasonic treatment to a biological entity. The ultrasound therapy device may include at least one waveform generator, one or more transducers, and a programmable controller. In some embodiments, the waveform generator is configured to generate a first set of drive signals and at least a second set of drive signals. In some embodiments, the first set of drive signals and the second set of drive signals comprise an average frequency independently selected from a range of greater than about 50 kHz (kilohertz) to less than about 4 MHz (megahertz).

The one or more transducers may be communicatively coupled (e.g., electrically, wirelessly, capacitively, or inductively coupled or combinations thereof to the waveform generator. In some embodiments, the one or more transducers are configured to receive the first drive signal and the second drive signal and to concurrently or sequentially generate a first ultrasonic signal and a second ultrasonic signal based on the first set of drive signals and the second set of drive signals. In some embodiments, the first ultrasonic signal comprises a spatial average-temporal average (SATA) intensity in the range of about 0.25 watts per square centimeter to about 3 watts per square centimeter, and the second ultrasonic signal comprises a spatial average intensity in the range of about 0.01 watts per square centimeter to about 0.25 watts per square centimeter.

The programmable controller may be communicatively coupled (e.g., electrically, wirelessly, capacitively, or inductively coupled or combinations thereof) to the waveform generator and/or to the one or more transducers. In some embodiments, the programmable controller is operable to provide the first set of drive signals and the second set of drive signals to the one or more transducers such that the one or more transducers operate in a pre-selected sequence, for a pre-selected period of time.

In another aspect, the present disclosure is directed to a method for providing thermal and non-thermal ultrasonic treatment to a subject. The method includes contacting a location on a biological interface of the subject with an ultrasound delivery device, the ultrasound delivery device comprising one or more ultrasound transducer. In some embodiments, the one or more ultrasound transducers are configured to provide thermally active moderate-intensity ultrasonic energy, and non-thermally active low-intensity ultrasonic energy. The method may further include applying a sufficient amount of current to the one or more ultrasound transducers to emit a therapeutically effective amount of the thermally active moderate-intensity ultrasonic energy and the non-thermally active low-intensity ultrasonic energy from the ultrasound delivery device.

In another aspect, the present disclosure is directed to an ultrasound therapy system for delivering thermally active moderate-intensity waveforms, and non-thermally active low-intensity waveforms. The system includes an ultrasound delivery device and a controller.

The ultrasound delivery device may include a first waveform generator configured to generate a first set of drive signals, and at least a second waveform generator configured to generate a second set of drive signals. The ultrasound delivery device further includes one or more transducers communicatively coupled (e.g., electrically, wirelessly, capacitively, or inductively coupled or combinations thereof to the first and/or at least second waveform generator. In some embodiments, the one or more transducers are configured to receive the first set of drive signals and the second set of drive signals and to concurrently or sequentially generate a first ultrasonic signal and a second ultrasonic signal based on the first set of drive signals and the second set of drive signals, the first ultrasonic signal having a spatial average intensity in the range of about 0.25 watts per square centimeter to about 3 watts per square centimeter, and the second ultrasonic signal having a spatial average-temporal average intensity in the range of about 0.01 watts per square centimeter to about 0.25 watts per square centimeter. In some embodiments, the controller is configured to communicate at least one instruction via electrical, wireless, or inductive communication to the first and the at least second waveform generators, and to the one or more transducers.

In yet another aspect, the present disclosure is directed to a method of therapeutic ultrasound treatment. The method includes generating a pulsed digital signal at a frequency in the range of greater than about 50 kHz to less than about 3.0 MHz, and a spatial average-temporal average intensity in the range of about 0.010 watts per square centimeter to about 3.0 watts per square centimeter. The method further includes converting the pulsed digital signal to a pulsed sine wave signal. The method may further include delivering the pulsed sine wave signal to a first plurality of separate locations via one or more transducers in a preselected sequence, each for a preselected time period. The method may further include receiving the pulsed sine wave signal at the first plurality of separate locations and delivering ultrasound energy to a biological entity from each of the separate locations while maintaining the first plurality of separate locations substantially stationary relative to a first area being treated. In some embodiments, the transducers are maintained substantially stationary during operation thereof.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1 is a top front isometric view of an exemplary ultrasound therapy system for delivering ultrasonic treatment to a biological entity according to one illustrated embodiment.

FIG. 2 is a top front isometric view of an exemplary ultrasound therapy system for delivering ultrasonic treatment to a biological entity according to one illustrated embodiment.

FIG. 3 is a top plan view of an exemplary ultrasound therapy system for delivering ultrasonic treatment to a biological entity according to another illustrated embodiment.

FIG. 4 is a schematic diagram of an ultrasound therapy device according to one illustrated embodiment.

FIGS. 5A and 5B are time versus amplitude plots of exemplary uni-variant and multi-variant waveforms according to multiple illustrated embodiments.

FIG. 6 is a schematic diagram of an ultrasound therapy device according to one illustrated embodiment.

FIG. 7 is a schematic diagram of an ultrasound therapy device in the form of one transducer according to one illustrated embodiment.

FIG. 8 is a schematic diagram of an ultrasound therapy device in the form of a plurality of transducers according to another illustrated embodiment.

FIG. 9 is a schematic diagram of an ultrasound therapy device in the form of a transducer including a plurality of piezoelectric crystals according to one illustrated embodiment.

FIG. 10 is a schematic diagram of an ultrasound therapy device in the form of a transducer including a plurality of piezoelectric crystals according to one illustrated embodiment.

FIGS. 11, 12, and 13 are top plan views of an ultrasound therapy system according to multiple illustrated embodiments.

FIGS. 14, 15, and 16 are schematic diagrams of an ultrasound therapy device according to multiple illustrated embodiments.

FIG. 17 is a schematic diagram of an inductively powered ultrasound therapy device according to one illustrated embodiment.

FIG. 18 is a flow diagram of a method of treating at least one condition associated with injured tissue in a subject according to one illustrated embodiment

FIG. 19 is a flow diagram of a method for providing thermal and non-thermal ultrasonic treatment to a subject according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with ultrasound devices including, but not limited to, voltage and/or current regulators, waveform generators, transducers and the like have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment,” or “in some embodiments” means that a particular referent feature, structure, or characteristic described in connection with the embodiment is included in at least one embodiment. Thus, the appearance of the phrases “in one embodiment,” or “in an embodiment,” or “in another embodiment,” or “in some embodiments” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an ultrasound therapy device including “a transducer” includes a single transducer, or two or more transducers. It should also be noted that the term “or” is generally employed in its sense including “and/or” unless the content clearly dictates otherwise.

The terms “subject” or “biological subject” generally refer to, without limitation, any biological mass (e.g., tissue, cells, and the like), host, animal, vertebrate, or invertebrate, and includes fish, mammals, amphibians, reptiles, birds, and particularly humans.

The term “ultrasound” generally refers to, without limitation, sound with a frequency greater than about 20,000 Hz (hertz). For a given ultrasound source, the higher the frequency, the less the emerging ultrasound signal diverges. Sound at audible frequencies may spread out in all directions, whereas ultrasound signals are typically collimated. Ultrasound signals at frequencies greater than 800 kHz are sufficiently collimated to selectively expose a limited target area for physical therapy treatment. At frequencies less than about 800 kHz, the ultrasound signal's intensity is sufficiently low so as to be outside the range for physical therapy treatment, but ultrasound has been used at these low intensity levels for diagnostic procedures.

Absorption of ultrasound, and therefore attenuation, increases as the frequency increases. Absorption occurs in part because of the internal friction in tissue that needs to be overcome in the passage of sound. The higher the frequency, the more rapidly the molecules are forced to move against this friction. As the absorption increases, less sound energy is available to propagate through the tissue. At frequencies greater than 20 MHz, superficial absorption may become so great that less than 1 percent of the sound penetrates beyond the first centimeter.

Generally, for physical therapy applications, the frequency range is often limited to frequencies within the range of about 800 kHz to about 3.3 MHz. In some instances, physical therapy applications employ a frequency of about 1 MHz or about 3 MHz because these frequencies offer a good compromise between sufficiently deep penetration and adequate heating under customary exposure levels.

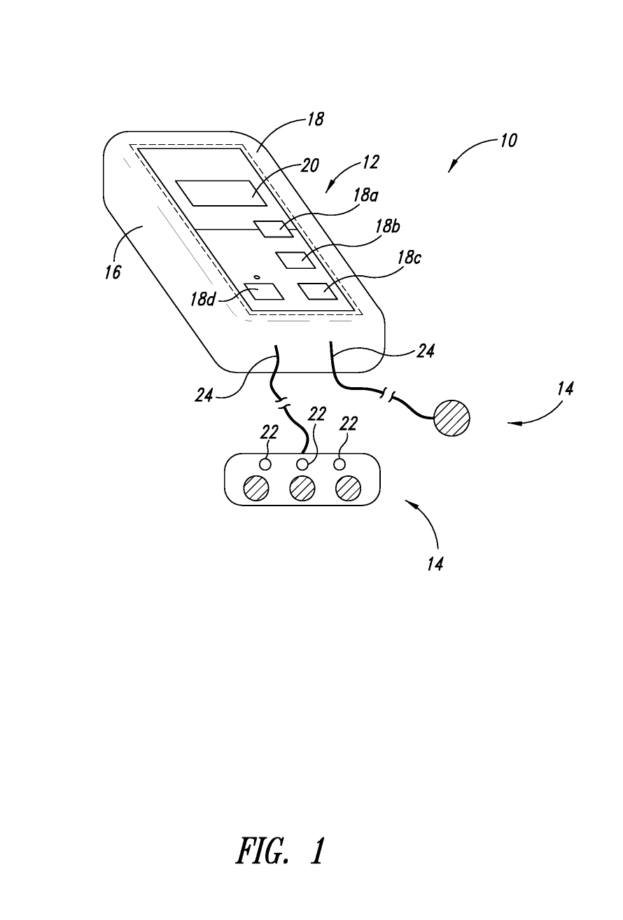

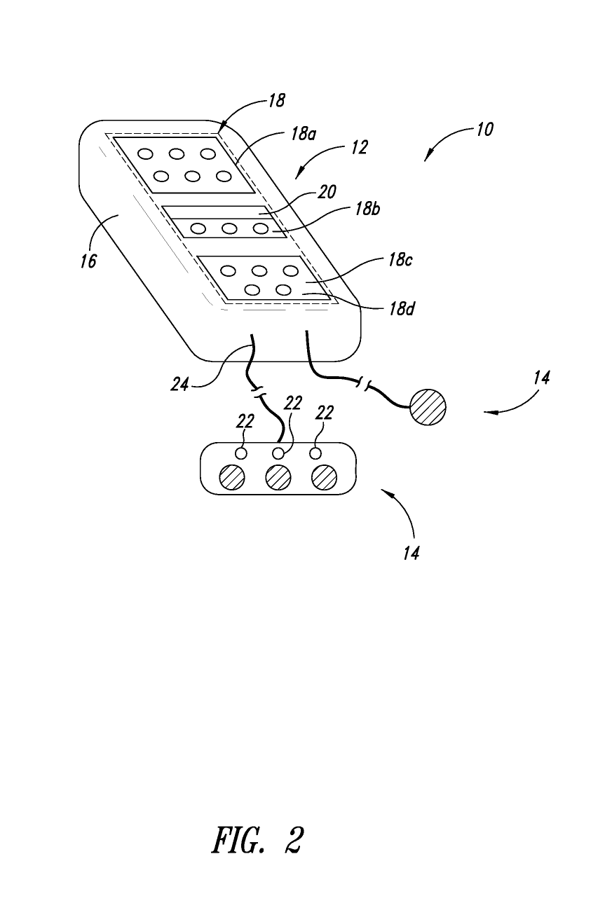

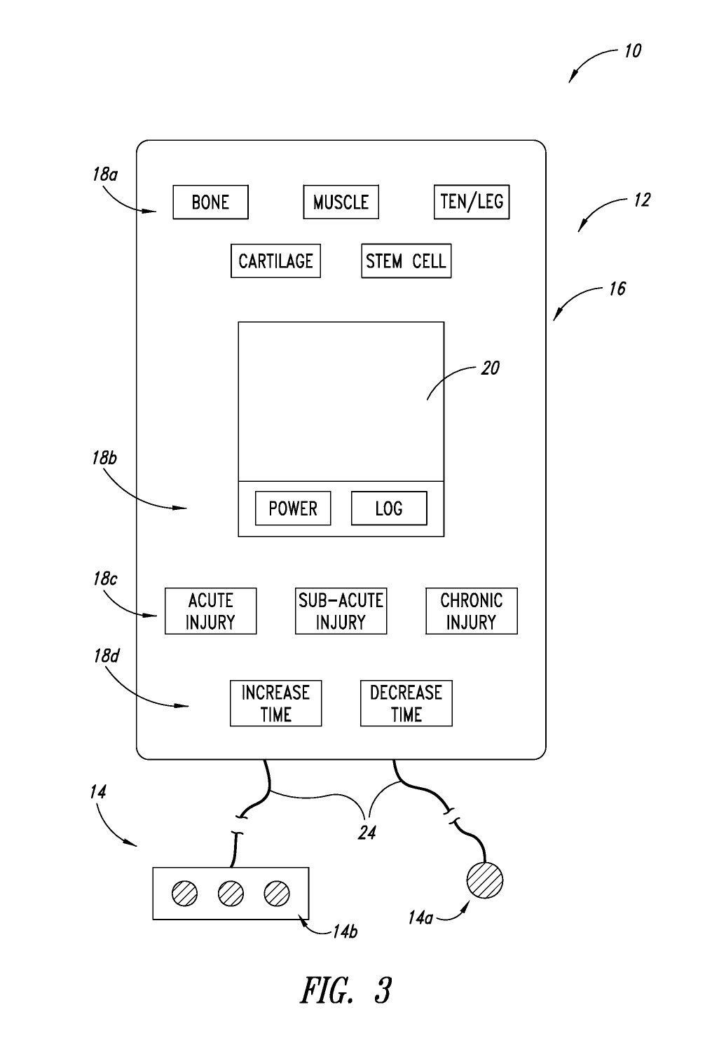

FIGS. 1, 2, and 3 show an exemplary ultrasound therapy system 10 for delivering ultrasonic treatment to a biological entity according to one illustrated embodiment. The system 10 includes an ultrasound therapy device 12 including a transducer 14.

The transducer 14 may include a housing, one or more transducer treatment elements, as well as any associated hardware or software. The transducer 14 may further include a system of transducers or individual transducer components. For example, the transducer 14 may take the form of a single transducer 14a, a plurality of transducers (a three transducer set up is depicted in 14b), and/or one or more arrays of transducers. In some embodiments, the ultrasound therapy device 12 may include at least a single transducer 14a and plurality of transducers 14b.

The transducer 14 may comprise a single element or multiple elements arranged in any spatial grouping desired. Further, the transducer 14 may be constructed in such a way as to have a single resonant frequency or multiple resonant frequencies, thereby affecting the frequency of the ultrasonic output waves. The transducer 14 may be of single design where a single piezoelectric crystal outputs one single waveform at a time, or may be compound where two or more piezoelectric crystals are utilized in a single transducer 14 or in multiple transducers 14 thereby allowing for multiple waveforms to be output concurrently.

The system 10 may be operable to concurrently utilize multiple transducer treatment elements. In some embodiments, the system 10 may include multiple drive circuits (e.g., one drive circuit for each transducer 14) and may be configured to generate varying waveforms from each coupled transducer (e.g., multiple waveform generators, and the like).

In some embodiments, the system 10 is configured to deliver non-thermally active low-intensity waveforms, and thermally active moderate-intensity waveforms.

Non-thermal waveforms are those waveforms that increase a target tissue temperature no more than about 1° C. and/or that elicit a non-thermal effect in a biological subject. Examples of non-thermal effects include an increase in blood flow, cell membrane permeability, fibroblastic activity, vascular permeability, and the like. Further examples of non-thermal effects include altered rates of diffusion across cell membranes, diffusion of ions, production of granulation tissue, secretion of chemotactics, stimulation of phagocytosis, synthesis of protein, tissue regeneration, decreased edema, and the like. Indications for non-thermal ultrasound therapy include acute injuries, subacute injuries, bursitis, tendonitis, chronic open wounds, sprains, strains, neuromas, and the like. Some of the mechanisms of action behind the non-thermal effects of therapeutic ultrasound, such as cavitation and acoustic streaming, have been shown to cause the up-regulation of many cellular processes. Indications for non-thermal ultrasound therapy include bone healing, chronic inflammation, pain reduction, scar tissue contracture, joint contracture, and the like.

Thermally active waveforms are those resulting in a target tissue temperature increase of about 1° C. or greater, and/or that elicit a thermal effect in a biological subject. In some embodiments, the target tissue temperature increase ranges from about 1° C. to about 4° C. Examples of thermal effects may include an increase in blood flow, collagen deposition, extensibility of structures (e.g., collagen), macrophage activity, motor nerve conduction velocity, sensory nerve conduction velocity, and the like. Further examples of thermal effects include a decrease in joint stiffness, muscle spasm, pain, and the like, as well as a mild inflammatory response that may enhance adhesion of leukocytes to damaged endothelial cells. Thermal effects may further include one or more of the non-thermal effects.

Research into the non-thermal effects of therapeutic ultrasound has uncovered other mechanisms of action, such as cavitation and acoustic streaming, which have been shown to cause the up-regulation of many cellular processes. These non-thermal effects of therapeutic ultrasound have been found to improve healing time and quality of healing in a wide variety of tissue types. For example, low-intensity therapeutic ultrasound has been shown to produce these same beneficial, non-thermal effects without creating an appreciable temperature increase in the treated tissues.

The strength of an ultrasound signal may be determined by its “intensity”. The term “intensity” generally refers to, without limitation, the rate at which energy is delivered per unit area, generally expressed in units of watts per square centimeter. Generally, ultrasonic intensity is measured and expressed in units of temporal average intensity; however, the most biologically relevant measure of ultrasonic energy deposition is expressed as Spatial Average-Temporal Average (SATA) intensity. While different expressions of ultrasonic energy delivery and absorption may be converted between one another by simply knowing the waveform characteristics and the surface area of the transducer, expressions of intensity herein are meant to represent Spatial Average-Temporal Average (SATA) intensity values. Intensities employed in physical therapy have generally been limited to the range of about 0.25 watts to about 3 watts per square centimeter. For pulsed ultrasound signals, the intensity of the signal is about zero when the ultrasound signal is OFF and at its maximum during the ON pulse. The temporal average intensity of a signal is obtained by averaging the intensity over both the ON and OFF periods. The amount of heating depends on the temporal average intensity. The temporal average intensity decreases proportionally with the amount of time the ultrasound signal is OFF. Thus, less heating will occur even though the temporal peak intensity is unchanged.

Because the ultrasound signal is often not uniform, some regions of the signal will be more intense than other regions. The measurement of intensity gives an average intensity and is referred to as the spatial average intensity. The term “spatial average intensity” generally refers to, without limitation, the amount of ultrasonic energy delivered by area of the transducer producing ultrasonic waves. Spatial average intensity is often expressed in watts per square centimeter (W/cm²). The World Health Organization limits the spatial average intensity to a maximum of 3 watts per square centimeter. Surgical tissue destroying techniques generally employ ultrasonic waves having intensities greater than 10 watts per square centimeter. Diagnostic applications generally used ultrasonic waves having intensities below 0.21 watts per square centimeter (temporal average).

Types of ultrasonic waves include low intensity, non-thermal waves; moderate intensity, thermally active waves; and high intensity, thermally ablative waves. The classifications of these ultrasonic waves can be understood most basically by considering the output ultrasonic wave's intensity alone. The total energy delivered over a prescribed area, during a specified time, however, is truly what determines the classifications of the ultrasonic waves.

As note previously, high intensity, thermally ablative waves are often utilized in ultrasonic scalpels and tissue ablation procedures. The intensities delivered by these waves are generally above 10 watts per square centimeter and are often well outside of the therapeutic region of interest.

Low intensity waves are those delivering intensities below 250 milliwatts per square centimeter. These waves may also be considered non-thermal waves since intensities below 250 milliwatts are generally incapable of eliciting an appreciable rise in target tissue temperature, especially when utilized in a pulsed fashion. In some embodiments, low intensity ultrasonic output waves are applied with the transducer 14 attached to the patient in a stationary fashion. Low intensity ultrasonic output waves are generally administer to a subject in a pulsed fashion, and may help speed the healing of various tissue types by up to 38%. Low-intensity ultrasound having intensities as low as about 0.015 watts per square centimeter may be effective at speeding the healing of a diverse group of tissues. Low-intensity ultrasound therapy produces therapeutic effects without an appreciable rise in target tissue temperature. Low-intensity therapeutic ultrasound waves are generally utilized in a pulsed application with intensities in the 0.03 watts to about 0.25 watts per square centimeter (SATA) range.

Unlike moderate intensity ultrasound therapy, low intensity ultrasound treatment enables the static placement of the treatment element on the subject without thermally damaging the target tissue. Accordingly, some embodiments of the disclosed systems, methods, and devices may be easier to use and may result in improving patient compliance.

Moderate intensity waves are those delivering intensities in the 250 milliwatts to 8 watts range. These waves are the most commonly applied ultrasonic waves in therapeutic ultrasound treatments. At intensities approaching 8 watts, most subjects perceive heat and pain. Therefore, these moderate intensity waves are most commonly employed in the range of about 0.5 watts to about 3 watts range, and may be applied in a continuous or pulsed fashion, with the continuous application delivering more total energy and, therefore, producing a more substantial rise in target tissue temperature. The therapeutic output waves may be delivered with the transducer treatment head kept in continual motion over an area roughly twice that of the damaged tissue. This movement of the treatment head over a large treatment area may act as a mechanical modulation of the total energy delivered.

The system 10 may further include a housing 16, a control module 18, and a display 20. The system 10 may also include one or more sensors 22 operable to determine at least one physiological characteristic of a biological entity. Examples of a physiological characteristic include, for example, an impedance, a temperature, a density, a vital statistic (e.g., a blood pressure, a pulse, or the like), and/or a fat content, and the like. Other physiological characteristics can also be determined.

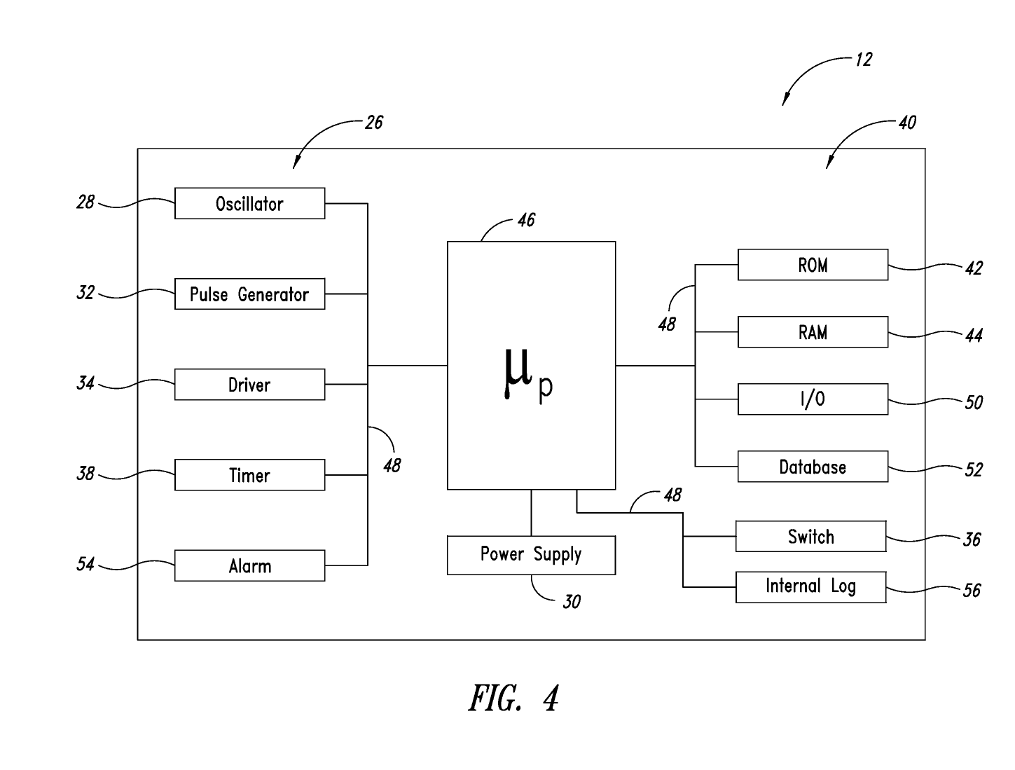

As shown in FIG. 4, the ultrasound therapy device 12 may further include at least one waveform generator 26, a controller system 40, and a power supply 30.

The term “set of drive signals” generally refers to, without limitation, electrical signals being directly or indirectly transferred from a waveform generator to a transducer. In some embodiments, the waveform generator 26 may include an oscillator 28 and a pulse generator 32 operable to generate a first set of drive signals and/or at least a second set of drive signals. In some embodiments, the oscillator 28 takes the form of a radio frequency (RF) oscillator operable to provide an RF signal to the transducer 14 causing the transducer 14 to ultrasonically vibrate and generate ultrasonic energy. The ultrasonic energy is subsequently transmitted to the injured tissue.

The waveform generator 26 may be configured to generate a first set of drive signals based in part on a user input. In some embodiments, the waveform generator 26 may be configured to generate at least a second set of drive signals based in part on a user input. The user input may include selections or signals indicative of one or more ultrasound characteristics, for example, at least one of an intensity, a frequency, a pulse ratio, a pulse intensity, a pulse duration, a pulse frequency, a pulse repetition rate, a continuous waveform frequency, a continuous waveform intensity, and/or one or more therapeutic characteristics, for example a treatment type, a treatment time, a treatment duration, a treatment time increase or decrease, a treatment interval rate, a lesion depth, a degree of tissue injury, a tissue type, and the like, or combinations thereof. The waveform generator 26 may further include a single oscillator 28 (e.g., variable frequency RF) programmed to deliver one or more frequencies based in part on the user input. In some other embodiments, the waveform generator 26 may include a plurality of oscillators 28, one for each type of tissue. In such embodiments, each of the oscillators 28 is pre-programmable to transmit a specific signal based in part on the tissue type associated with the particular oscillator 28. The oscillator 28 is operable to generate a signal with a frequency ranging from about 20 kHz to about 3 MHz.

In some embodiments, the system 10 may include two or more waveform generators 26 operable to produce low-intensity ultrasound waveforms, moderate intensity ultrasound waveforms, or combinations thereof.

Ultrasound waves can be produced as a continuous wave, a pulsed wave, or combinations thereof. In some embodiments, a pulsed wave is intermittently interrupted. The pulse generator 32 may generate pulsed periods and non-pulsed (or inactive) periods. Pulsed waves may be characterized by specifying the fraction of time the ultrasound is present over one pulse period. This fraction is called the duty cycle and is calculated by dividing the pulse time ON by the total time of a pulse period (e.g., time ON plus time OFF). For example, in some embodiments, the pulse generator 32 may be configured to electronically generate pulsed periods and non-pulsed (or inactive) periods.

A pulse ratio refers to the ratio of time that the pulse generator 32 is active (generating pulsed periods) to the time that the pulse generator 32 is inactive (generating non-pulsed periods). Similarly, a duty cycle refers to a ratio of a pulse signal duration relative to a pulse signal period. For example, a pulse signal duration of 10 μs and a pulse signal period of 20 μs, corresponds to a duty cycle of 0.5. Duty cycles, when in the pulsed mode, may range from about 0.05 (5%) to about 0.5 (50%).

In some embodiments, the system 10 is adapted to provide the stationary application of the transducers 14 during use. The system 10 is operable to generate a pulsed digital signal at a frequency in the range of greater than about 50 kHz to less than about 4.0 MHz and a spatial average-temporal average intensity in the range of about 0.010 watts per square centimeter to about 3.0 watts per square centimeter.

In some embodiments, the system 10 is operable to provide a first ultrasonic signal having a spatial average-temporal average intensity in the range of about 0.25 watts per square centimeter to about 3 watts per square centimeter, and at least a second ultrasonic signal having a spatial average-temporal average intensity in the range of about 0.01 watts per square centimeter to about 0.25 watts per square centimeter.

In some embodiments, the pulse generator 32 generates pulsed and non-pulsed periods in a pulse ratio dependent on the type of injury. In general, however, the pulse ratio ranges from about 1:1 to about 1:20. In some embodiments, the pulse ratio ranges from about 1:1 to about 1:8. In one exemplary embodiment, for chronic injuries, the pulse ratio ranges from about 1:1 to about 1:2, and for acute injuries, the pulse ratio ranges from about 1:3 to about 1:4. Other pulse ratios may also be possible.

The pulse generator 32 varies the intensity of the pulses depending, in part, on the type of tissue and injury selected. In some embodiments, the pulse generator 32 may be configured to, for example, electronically vary the intensity of one or more pulses depending, in part, on the type of tissue and injury selected. For example, for chronic injuries, the pulse generator 32 outputs a pulse having a greater intensity than the pulse output for acute injuries. Although dependent on the type of injury and tissue, the pulse generator 32 is generally operable to vary the intensity of a pulse from about 10 mW/cm² to about 3 W/cm².

In the case of two or more waveform generators 26, each generator waveform 26 may alternatively or concurrently provide low- and moderate-intensity waves. In some embodiments, the system may deliver low intensity or moderate intensity waves in a continuous fashion for a period of time of about 3 to about 10 minutes (e.g., to cause heating) and then switch the transducers 14 to deliver fixed, low-intensity waveforms or multi-variant, low-intensity waveforms (as described herein). In some embodiments, programmed waveform applications may be overridden by subject sensors (especially temperature) to be certain that tissues are not over heated.

In some embodiments, one or more continuous waveform segments may be combined with the generated pulsed periods and/or non-pulsed (or inactive) periods.

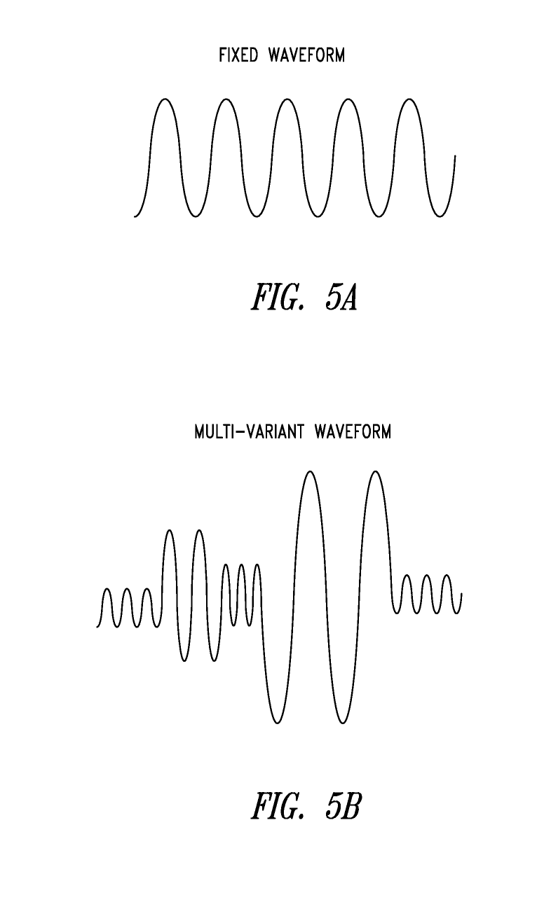

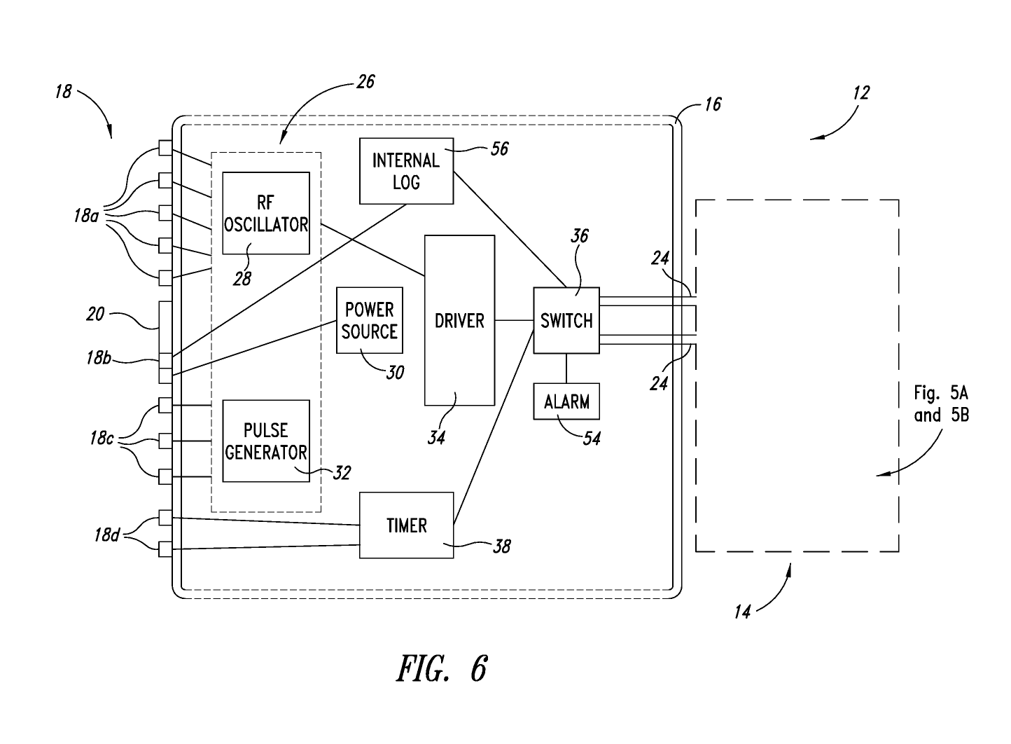

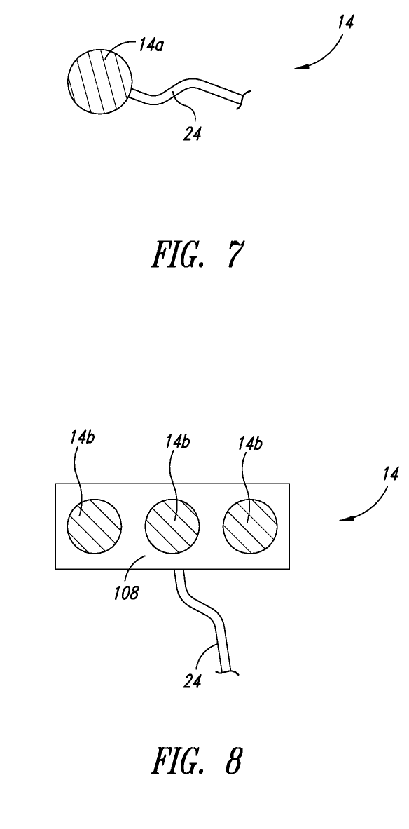

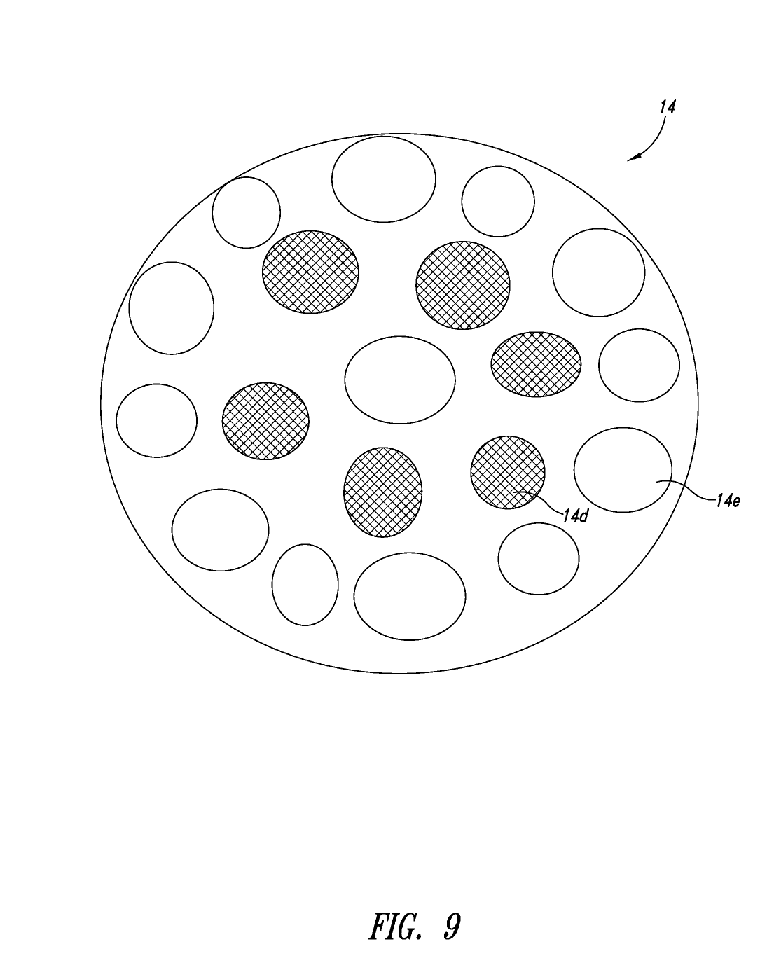

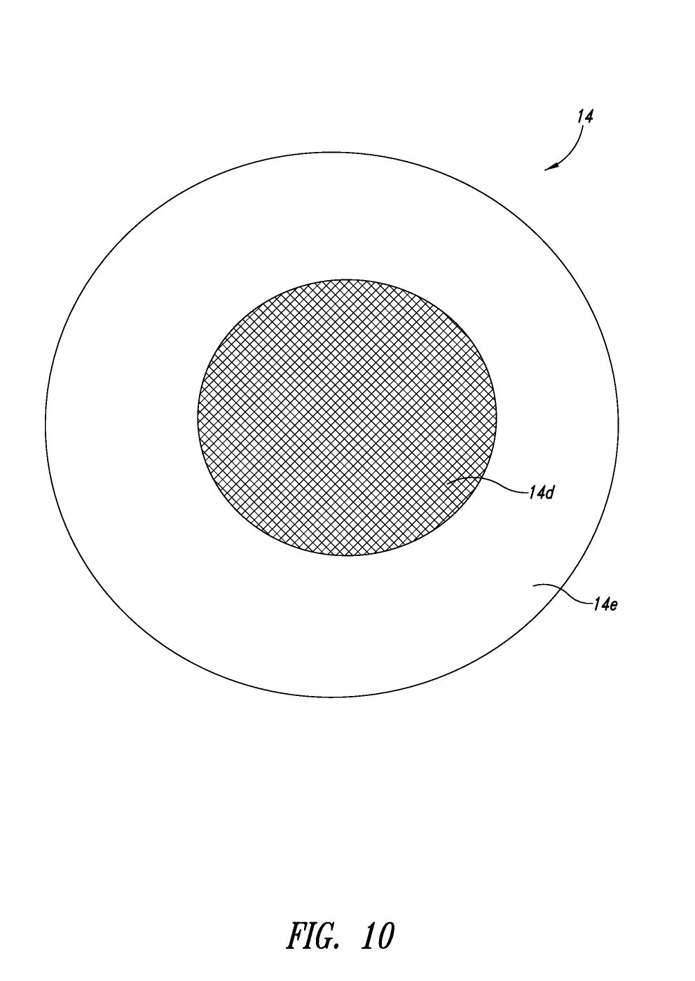

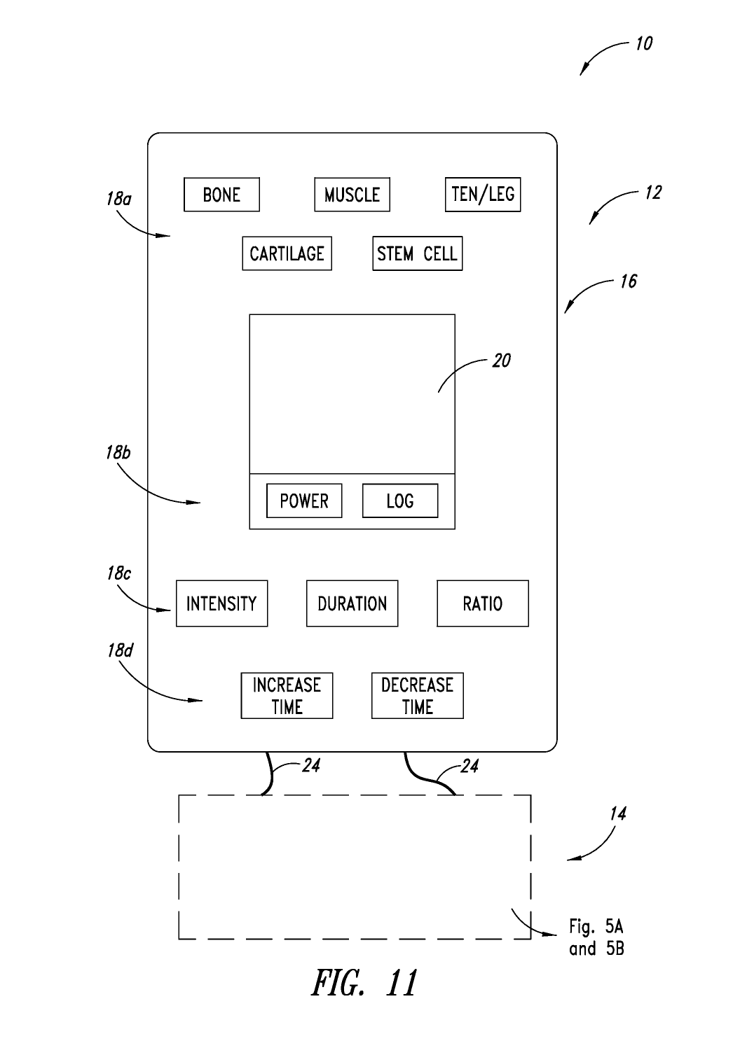

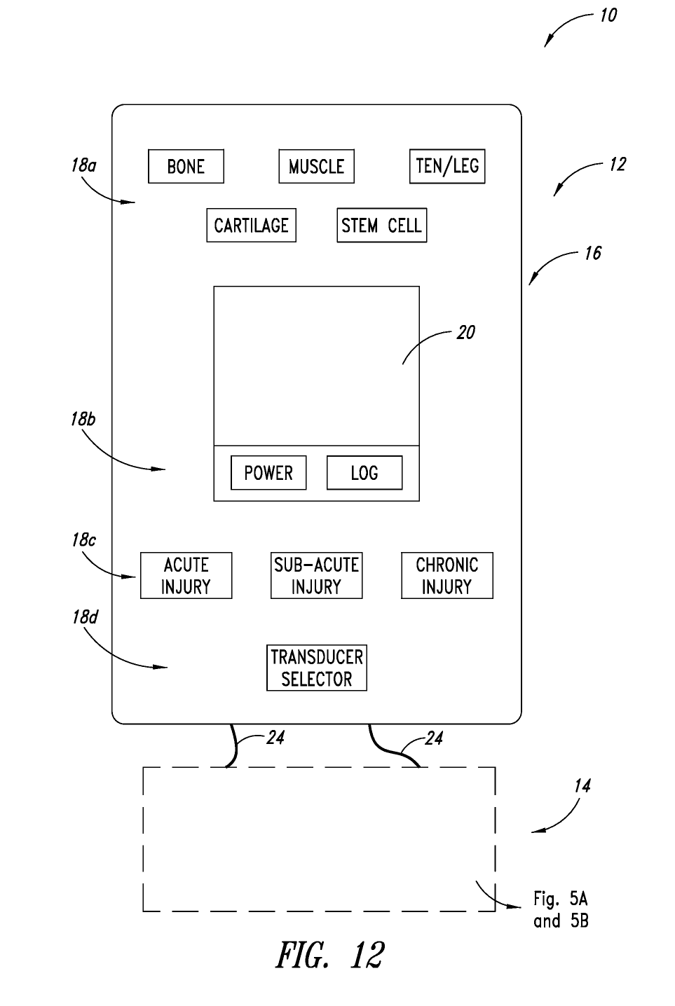

Ultrasound therapy may be provided in one or more treatment segments. In some embodiments, one or more waveform segments may be combined to form a drive signal train. In some embodiments, the waveform of each treatment segment may differ from the preceding or subsequent waveform segment in at least one waveform characteristic (e.g., intensity, frequency, pulse intensity, pulse duration, pulse ratio, pulse repetition rate, and the like). Waveforms having segments that vary in only one characteristic are generally said to be uni-variant (see e.g., FIG. 5A), and waveforms having segments that vary in more than one characteristic are generally said to be multi-variant (see e.g., FIG. 5B). Waveforms with constant amplitude and frequency are generally said to be continuous waveforms. Continuous waveforms may vary in intensity and/or frequency.

By utilizing the concept of multiple treatment segments employing uni-variant and/or multi-variant output waveforms, it has become possible to utilize both low intensity, non-thermal waveforms and moderate intensity, thermally active waveforms in a sequential or concurrent fashion. This concept allows for this treatment to occur with the transducers placed in a substantially stationary fashion on the treatment subject.

During treatment, the ultrasound device 12 may cycle through the selected waveform segments at selected times to treat the selected tissue type until all the waveforms are used and/or the treatment time elapses. This cycling of varying waveform segments enables the safe application of therapeutic ultrasound through the generally static placement of transducer treatment elements. The varying waveform modulates and controls the power density received by the target tissue throughout the treatment and maintains the power density within optimal therapeutic levels. In some embodiments, the ultrasound device 12 may cycle through one or more uni-variant, multi-variant, and continuous waveforms, or combinations thereof. In some embodiments, the intensity and/or frequency of the waveforms may be electronically modulated.

In some embodiments, the pulse generator 32 may vary the intensity of a single pulse or a series of pulses throughout a single treatment session. For example, the pulse generator 32 may vary the intensity of a single pulse in a series of pulses delivered in a single treatment session such that each pulse outputted by the pulse generator 32 has a multi-variant waveform. The pulse generator 32 may also vary the intensity of the series of pulses, such that each pulse in the series of pulses delivered in a single treatment session has a uni-variant waveform. The pulse generator 32 may be operable to vary the intensity of each pulse and the intensity of the series of pulses delivered in a single treatment session.

The generated pulses may take a variety of forms including multi-variant waveforms, uni-variant waveforms, continuous waveforms, or combinations thereof. In some embodiments, one or more pulses in a pulse series may have uni-variant waveforms and one or more pulses in the pulse series may have multi-variant waveforms. In some further embodiments, the generated pulses may include one or more continuous waveforms segments or pulsed waveforms with fixed waveform characteristics.

The frequency needed to reach each tissue type is generally known in the relevant art. The pulse generator 32 may control the intensity, frequency, pulse intensity, duration, ratio, and/or repetition rate, each of which is determined based in part on the injury type and the tissue type.

Depending on the injury type, the pulse generator 32 generally generates a pulse having a pulse duration ranging from about 10 μs to about 2,500 μs, a pulse repetition rate (frequency) ranging from about 50 Hz to about 10,000 Hz and a pulse intensity ranging from about 10 mW/cm² to about 3 W/cm². In one exemplary embodiment, the pulse frequency is about 1,000 Hz and the pulse duration ranges from about 100 μs to about 400 μs.

In some embodiments, the ultrasound therapy device 12 may include one or more waveform generators 26 configured to generate a first set of drive signals and at least a second set of drive signals. In some embodiments, the first set of drive signals and the second set of drive signals comprise an average frequency independently selected from a range of greater than about 50 kHz to less than about 4 MHz. In some embodiments, the first set of drive signals comprises a continuous or pulsed digital signal, and the at least second set of drive signals comprises a continuous or pulsed digital signal.

The one or more transducers 14 may be communicatively coupled to the waveform generator 26. In some embodiments, the one or more transducers 14 are configured to receive the first set of drive signals and the at least second set of drive signals and to concurrently or sequentially generate a first ultrasonic signal and at least a second ultrasonic signal based on the first set of drive signals and the at least second set of drive signals. In some embodiments, the first ultrasonic signal comprises a spatial average-temporal average intensity in the range of about 0.25 watts per square centimeter to about 3 watts per square centimeter, and the second ultrasonic signal comprises a spatial average-temporal average intensity in the range of about 0.01 watts per square centimeter to about 0.25 watts per square centimeter; and

In some embodiments, the first set of drive signals comprises at least a first waveform segment and a second waveform segment different from the first waveform segment; and the second set of drive signals comprises at least a third waveform segment and a fourth waveform segment different from the third waveform segment. The second waveform segment may have at least one of an intensity, a frequency, a pulse intensity, a pulse duration, a pulse frequency, a pulse ratio, or a pulse repetition rate different from the first waveform segment; and the fourth waveform segment may have at least one of an intensity, a frequency, a pulse intensity, a pulse duration, a pulse frequency, a pulse ratio, or a pulse repetition rate different from the third waveform segment.

In some embodiments, the one or more transducers 14 are configured to receive the first set of drive signals and the second set of drive signals and to generate a first non-thermal ultrasonic waveform and a second thermal ultrasonic waveform based on the first set of drive signals and the second set of drive signals. In some embodiments, the one or more transducers 14 are configured to receive the first set of drive signals and the second set of drive signals and to concurrently or sequentially generate a first non-thermal ultrasonic waveform and a second thermal ultrasonic waveform based on the first set of drive signals and the second set of drive signals.

Depending on the input (e.g., the injury type, the lesion depth, the degree of tissue injury, the tissue type, and the like) the waveform generator 26 may generate a first signal, or at least a second signal, having varying characteristics including, for example, the length of each waveform segment. In some embodiments, the waveform generator 26 is operable to generate continuous waveforms, pulsed waveforms, or combinations thereof. For example, in some embodiments, the waveform generator 26 may generate a first signal, or at least a second signal, having varying characteristics including, for example, varying intensities, frequencies, pulse intensities, pulse durations, pulse ratios, and/or pulse repetition rates for each waveform segment, based in part on the input. In some embodiments, the first waveform segment has at least one of an intensity, a frequency, a pulse intensity, a pulse duration, a pulse frequency, a pulse ratio, or a pulse repetition rate different from the second waveform segment. In some embodiments, the first and the second waveform segments are each selected from one or more single-sine waveforms, multi-sine waveforms, frequency-swept sine waveforms, step waveforms, pulse waveforms, square waveforms, triangular waveforms, saw-tooth waveforms, arbitrary waveforms, generated waveforms, chirp waveforms, non-sinusoidal waveforms, and ramp waveforms, or combinations thereof including, for example, single and multi-frequency formed waves. In some embodiments, the ultrasound therapy device 12 may further be configured to cycle through the at least first and second waveform segments for a limited treatment time. In some embodiments, the generated first ultrasonic signal compromise at least one of a continuous or a pulsed ultrasonic treatment wave, or combinations thereof. In some embodiments, the ultrasound therapy device 12 may be further configured to electronically modulate at least one of an intensity, a frequency, a pulse intensity, a pulse duration, a pulse frequency, a pulse ratio, or a pulse repetition rate of a waveform associated with the first drive signal. In some embodiments, the generated second ultrasonic signal compromise at least one of a continuous or a pulsed ultrasonic treatment wave, or combinations thereof. In some embodiments, the ultrasound therapy device 12 may be further configured to electronically modulate at least one of an intensity, a frequency, a pulse intensity, a pulse duration, a pulse frequency, a pulse ratio, or a pulse repetition rate of a waveform associated with the second drive signal.

In some embodiments, the intensity of the first or second ultrasonic signal ranges from about 0.01 W/cm² to about 3 W/cm². In some embodiments, the intensity of the first or second ultrasonic signal ranges from about 0.01 W/cm² to about 1.5 W/cm². In some embodiments, the intensity of the first or second ultrasonic signal ranges from about 0.4 W/cm² to about 1.5 W/cm². In some embodiments, the pulse ratio of the first or second ultrasonic signal ranges from about to about 1:1 to about 1:20. In some embodiments, the frequency of the first or second ultrasonic signal ranges from about 0.05 MHz to about 3 MHz. In some embodiments, the pulse repetition rate of the first or second ultrasonic signal ranges from about 500 Hz to about 2500 Hz. In some embodiments, the pulse repetition rate of the first or second ultrasonic signal ranges from about 50 KHz to about 10,000 Hz. In some embodiments, the pulse duration of the first or second ultrasonic signal ranges from about 10 μs to about 2,500 μs. In some embodiments, the first or second ultrasonic signal comprises a pulse duration ranging from about 10 μs to about 2,500 μs, a pulse ratio ranging from about 1:1 to about 1:8, a pulse repetition rate ranging from about 50 Hz to about 10,000 Hz, and a pulse intensity ranging about 0.01 W/cm² to about 3 W/cm². In some embodiments, the first or second ultrasonic signal comprises a pulse repetition rate ranging from about 500 Hz to about 2,500 Hz, and a pulse duration ranging from about 100 μs to about 500 μs.

Referring to FIG. 4, the therapy device 12 may include one or more controllers 46 such as a microprocessor, a digital signal processor (DSP) (not shown), an application-specific integrated circuit (ASIC) (not shown), field programmable gate array (FPGA), and the like and may include discrete digital and/or analog circuit elements or electronics.

In some embodiments, one or more controllers 46 take the form of an intensity modifying circuit operable to dampen or boost one or more signal generated by the waveform generator 26. In some embodiments, the intensity modifying circuit is operable to dampen or boost the intensity of a first set of drive signals and at least a second set of drive signals such that some of the one or more transducers 14 produce waveforms of only moderate intensity or low-intensity, and the intensity modifying circuit operable to dampen or boost the intensity to our desired low-intensity (or moderate intensity) waveforms and delivers the modified waves to approximately half of the transducers.

The device 12 may also include a control system 40 for selectively controlling various aspects of the ultrasound therapy device 12. The control system 40 may include one or more memories that store instructions and/or data, for example, read-only memory (ROM) 42, random access memory (RAM) 44, and the like, coupled to the controller 46 by one or more busses 48. The control system 40 may further include one or more input devices 50 including, for example, a display 20, a controller module 18 including one or more treatment controller modules 18a, 18b, 18c, 18d, and the like, or any peripheral device. In some embodiments, the controller 46 is configured to compare a physiological characteristic of a biological entity to a database 52 of stored reference values, and to generate a response based in part on the comparison. For example, the controller 46 may be configured to compare a measured impedance, temperature, density, or fat content of a biological entity to a database 52 of stored reference values, and to generate a response based in part on the comparison. The database 52 of stored values may include characteristic physiological data including, for example, characteristic impedance data, characteristic temperature data, characteristic density data, characteristic fat content data, characteristic treatment delivery data, and the like. In some embodiments, the generated response includes at least one of a response signal, a comparison plot, a treatment code, a diagnostic code, a test code, an alarm, a change to a treatment parameter, a response signal operable to terminate the first drive signal, and the like.

In some embodiments, the control system 40 includes a programmable control element communicatively coupled (e.g., electrically, wirelessly, capacitively, or inductively coupled or combinations thereof) to the waveform generator 26 and the one or more transducers 14. In some embodiments, the programmable control element is operable to provide the first set of drive signals and the second set of drive signals to the one or more transducers 14 such that the one or more transducers 14 operate in a selected sequence, for a selected period of time. As no two transducers are identical, the driving voltage necessary to output a desired amount of ultrasonic energy will differ between transducers. In some embodiments, the control system 40 may be configured to select the proper amount of voltage necessary to drive each transducer 14 such that the ultrasonic output is of the desired intensity. This may be accomplished by impedance measurements or voltage drain measurements of the individual transducers or the like. Alternatively, each transducer may be “tuned” prior to device manufacturing such that the specific voltage necessary to deliver a desired ultrasonic output intensity is accurately measured and established. This information is then made integral to each transducer by placement of an identification circuit, chip or the like on the transducer backing, housing pigtail cable, or the like. The control system 40 then reads the identification circuit and delivers the proper amount of energy to each transducer.

In some embodiments, the control system 40 may be configured to modulate at least one of an intensity, a frequency, a pulse intensity, a pulse duration, a pulse frequency, a pulse ratio, or a pulse repetition rate of a waveform associated with the first set of drive signals, the second set of drive signals, the first ultrasonic signal, and/or at least second ultrasonic signal. In some further embodiments, the control system 40 may be configured to electronically modulate at least one of an intensity, a frequency, a pulse intensity, a pulse duration, a pulse frequency, a pulse ratio, or a pulse repetition rate of a waveform associated with the first set of drive signals, the second set of drive signals, the first ultrasonic signal, and/or at least second ultrasonic signal.

As shown in FIG. 6, the waveform generator 26 may be communicatively coupled (e.g., electrically, wirelessly, capacitively, or inductively coupled or connected, or combinations thereof) to a driver 34 operable to modulate the output of the oscillator 28 in the form of an RF oscillator with the signal generated by the pulse generator 32 to generate a single signal. In one exemplary embodiment, the driver 34 generates a signal having a frequency ranging from about 50 kHz to about 3 MHz during pulsed periods. In some embodiments, the driver 34 also amplifies the resulting signal in order to deliver power having a maximum intensity for safe and effective ultrasonic therapy. In some embodiments, the driver 34 is operable to generate a signal selected from one or more single-sine waveforms, multi-sine waveforms, frequency-swept sine waveforms, step waveforms, pulse waveforms, square waveforms, triangular waveforms, saw-tooth waveforms, arbitrary waveforms, generated waveforms, chirp waveforms, non-sinusoidal waveforms, and ramp waveforms, or combinations thereof, including single and multi-frequency formed waves. In one exemplary embodiment, the power has a maximum intensity of about 3 W/cm². In another exemplary embodiment, the driver 34 is operable to deliver power having an intensity ranging from about 10 mW/cm² to about 500 mW/cm².

In some embodiments, the driver 34 is electrically or otherwise communicatively coupled to a switch 36. The switch 36 is electrically or otherwise communicatively coupled with one or more transducer cables 24, which electrically, wirelessly, capacitively, or inductively communicates with the transducer 14. In another exemplary embodiment, multiple cables 24 electrically communicate with the transducer 14. In use, the switch 36 receives a signal from the driver 34 and transmits the signal via cable 24 to the transducer 14. Referring to FIGS. 7 and 8, the transducer 14 may be a single transducer (as shown, for example, in FIG. 7), a plurality of transducers, and/or an array of transducers (as shown, for example, in FIG. 8).

Referring to FIGS. 9 and 10, in some embodiments, the disclosed devices and systems may include a single transducer 14, one or more transducers, or a compound transducer, as well as a multi-transducer treatment element; each capable of delivering both the low-intensity and the moderate-intensity waveforms concurrently. The transducers 14 may include multiple transducer elements 14d, 14e capable of generating both low and moderate intensity ultrasound waves. The grouping and number of transducers 14 and transducer elements 14d, 14e may vary with, for example, the size and shape depending on the application. The transducers 14 may be constructed in any spatial orientation such as a linear array or two-dimensional array. In some embodiments, transducer elements 14d may be operable to output low-intensity waveforms, while transducer elements 14e may be operable to output moderate-intensity waveforms. In some embodiments, transducer elements 14d, 14e may output a first type of waveform for a predetermined period of time and then switch to a second type (e.g., from moderate to low-intensity and vice-versa) of waveform for a subsequent predetermined period of time.

In some embodiments, the transducers 14 may include a plurality of spaced apart piezoelectric crystals 14d, 14e each operatively connected to the controller 46 for sequentially receiving a signal and delivering an ultrasound signal. The spaced apart piezoelectric crystals 14d, 14e may be grouped into at least two activation groups, one group 14d operable to receive first a signal for non-thermal waveform output for a determined time followed by a signal for a thermal waveform output for a determined time, the other group 14e operable to receive a signal for a thermal waveform output for a determined time followed by a signal for a non-thermal output for a determined time. In some embodiments, the spaced apart piezoelectric crystals 14d, 14e are spatially oriented such that no two adjacent piezoelectric crystals 14d, 14e are in the same activation group. The spaced apart piezoelectric crystals 14d, 14e may be spatially oriented such that no two adjacent piezoelectric crystals 14d, 14e are in the same activation group. At least one of the one or more transducers 14 may includes two or more spatially adjacent piezoelectric crystals 14d, 14e each piezoelectric crystal configured to generate either a thermal waveform or a non-thermal waveform.

In some embodiments, a programmable control element is connected to filtering element in the form of a sine filter adapted to deliver a signal to a plurality of separately-located transducers 14 in a selected sequence, each for a selected period of time.

A transducer 14 may include a plurality of spaced apart piezoelectric crystals 14d, 14e each operatively connected to the programmable control element for sequentially receiving a first and/or second signal and delivering a pulsed ultrasound signal.

The ultrasound therapy device 12 of FIG. 6 can further include a timer 38 for timing the treatment. An input device element (e.g., one or more selectors, buttons, and the like) of the control module 18 is electrically, wirelessly, capacitively, or inductively connected to the timer 38 for setting the treatment time. The input device element can have any construction suitable for setting the treatment time. For example, the input device element may comprise a button that alters the treatment time upon depression. Additionally, the housing 16 may include a touch-sensitive screen having user selectable icons or areas designated for time selection, for altering the treatment time upon touching the desired area of the screen, and the like. The input device element may be capable of scrolling, rotating, or otherwise altering the treatment time. In one exemplary embodiment, the treatment time ranges from about 5 minutes to about 60 minutes per session and from 1 to 3 sessions per 24-hour period. In another exemplary embodiment, the treatment time ranges from about 30 minutes to about 45 minutes per session. Daily treatments may continue until the injured tissue is partially or completely healed.

The timer 38 is communicatively coupled to the switch 36. Upon setting the timer 38 and starting a treatment session, the switch 36 transmits the signal from the driver 34 to the cable 24. When the treatment time has elapsed, the switch 36 ceases to deliver the signal from the driver 34 to the cable 24, thereby ending delivery of ultrasonic energy. In one embodiment, the timer is programmed with treatment times based in part on tissue and injury types. The timer may also include an override feature operable for setting treatment times outside the preset parameters.

In some embodiments, a controller 46, in the form of a microprocessor, associated with the control module 18 may determine in part the order in which the device 12 cycles through each treatment segment. The microprocessor may be factory pre-programmed or user accessible via a USB or other connection or by direct access from the device controls via, for example, a touch screen, rotating dial, and/or button push.

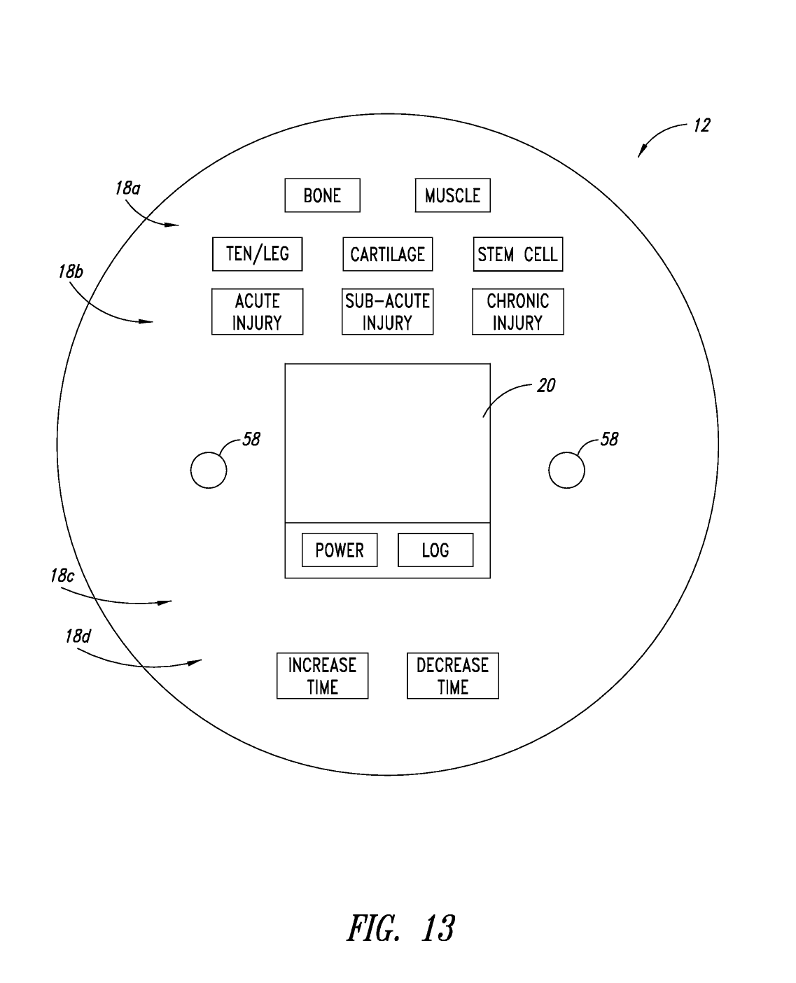

As shown in FIGS. 11, 12, and 13, the ultrasound therapy device 12 may include control module 18 including one or more treatment control modules 18a, 18b, 18c, 18d each including one or more input device elements for providing one or more treatment parameters. Examples of treatment parameters include, without limitation, a treatment type, a treatment time, a treatment duration, a lesion depth, a degree of tissue injury, a tissue type, an intensity, a frequency, a pulse ratio, a pulse intensity, a pulse duration, a pulse frequency, a pulse repetition rate, and the like.

The one or more treatment control modules 18a, 18b, 18c, 18d can generally be similar, or different and may have any construction suitable for providing input and/or output. The one or more treatment control modules 18a, 18b, 18c, 18d may also be operable to effect a user or treatment selection. For example, each treatment control module 18a, 18b, 18c, 18d may comprise an input device element in the form of a button, key or other input element for effecting a selection upon depression and or an output device in the form of a screen or a touch-sensitive screen having icons or areas designated for input and/or output. For example, the housing 16 may include a touch-sensitive screen 20 having icons or areas designated for inputting and/or outputting treatment control parameters and that, for example, effect selection or treatment input upon touching a desired area of the screen. The control module 18 may take the form of a single control module capable of scrolling, rotating or otherwise effecting selection and/or providing input/output capabilities. In one exemplary embodiment, the one or more treatment control modules 18a, 18b, 18c, 18d of control model 18 are adapted to enable input of tissue type, and/or degree of injury. The tissue type, as well as the depth at which the tissue lies within the body, is used to determine, in part, the ultrasonic frequency of the pulses generated by the device 12.

The control module 18 may be used to input or provide treatment parameters. For example, based on selections, programmed instruction, and/or inputs made by activating or engaging one or more input device elements (e.g., a treatment selection button, a touch-screen icon or area, and the like), a waveform (e.g., a first set of drive signals, and the like) is generated for providing treatment. The waveform generated may comprise a plurality of waveform segments, each segment having at least one characteristic different from the previous or subsequent waveform segment. In some embodiments, the different waveform segments may differ in one or more characteristics such as intensity, frequency, pulse ratio, pulse intensity, pulse duration, pulse repetition rate, and the like. In some embodiments, the ultrasound therapy device 12 is operable to cycle through the selected waveform segments. Each waveform segment is activated for a certain period of time before the device 12 cycles to the next waveform segment. The device 12 may continue to cycle through the waveform segments until all the segments have been used at least once, and/or the total treatment time elapses.

In some embodiments, the control module 18 may be programmed to deliver waveform characteristics based on the type of injury inputted, selected, preprogrammed, and/or provided. In one embodiment, the control module 18 may be pre-programmed by a manufacturer to enable and/or deliver a specific waveform based on the tissue selected. In some embodiments, one, some, or all of the treatment control modules 18a, 18b, 18c, and 18d may be included. The programmed treatment control module may include variations in waveform characteristics such as the length of each treatment segment and the cycling order of the treatment segments. Each treatment control module 18a, 18b, 18c, 18d may communicate with the waveform generator 26. In some embodiments, the control module 18 may include at least three input device elements, one each for chronic injuries, sub-acute injuries, and acute injuries. In some embodiments, the control module 18 includes one or more input device elements, one for each tissue type including bone, muscle, cartilage, tendons and/or ligaments, stem cells, and the like.

In some embodiments, a first input device element can control the pulse intensity, a second input device element can control the pulse duration, and a third input device element can control the pulse ratio. The combination of pulse duration and pulse ratio generates the pulse repetition rate. As such, the pulse repetition rate is automatically generated by the selection of a pulse duration and a pulse ratio. Because this fine-tuning of treatment variables requires extensive knowledge of therapeutic principles and medical practices, certain embodiment are designed for use by, for example, qualified operators.

An ultrasonic frequency can be first selected by tissue type through a plurality of input device elements. Pulse intensity may then be selected, since the selection of pulse intensity affects the available parameters for the remaining waveform characteristics. The input device elements can be pre-programmed to prevent selection of combinations of waveform characteristics and treatment segment times that may lead to thermal or other tissue damage. Once pulse intensity is selected, the pulse duration is selected. Next, the pulse ratio is selected, and finally, the treatment time is set. After setting the first waveform, the user may set another waveform, or simply begin treatment with a single waveform.

The device 12 can also include an alarm 54 (see FIGS. 4 and 6) for alerting the user of an event. For example, the alarm 54 can indicate a completion of a treatment portion, treatment completion, elapse of a treatment time, a malfunction, an error, and the like. The alarm 54 may be electrically or otherwise coupled to the switch 36 and configured to cease delivery of the first set of drive signals and/or second set of drive signals from the driver 34 to the cable 24, and instead energize the alarm 54, which alerts the user to, for example, the completion of the treatment. The alarm 54 may alert the user by, for example, an audible alarm that rings or otherwise makes a noise indicating the completion of the treatment, and the like. Additionally or alternatively, the alarm may alert the user by flashing lights, and or displaying a code, a message, an instruction, and the like and/or producing a tactile sensation.

The switch 36 may also comprise an interrupt feature for pausing treatment, for example, either when a loss of contact between the transducer 14 and the treatment area occurs or when the transducer 14 is otherwise not functioning properly. When such an event takes place, the switch 36 will cease delivering the signal from the driver 34 to the transducer 14, and will energize the alarm 54 to alert the user to the malfunction or interruption.

The device 12 may further include at least one display screen 20 and an internal log 56 for documenting and/or tracking one or more treatment variables including, for example, a usage of the device 12 and treatment specifics. In addition, the device 12 may be operable for allowing the entering of, for example, patient data. For example, the control module 18 may include an input screen, a keyboard, keypad, barcode, RFID or magnetic strip reader or the like for entering patient data, such as the patient's name, age, weight, injury complained of, and the like. The display screen 20 can be any suitable screen for displaying and/or inputting the desired information, for example a liquid crystal display screen. Additionally, at least two display screens 20 can be provided, one for displaying information related to treatment specifics, and one displaying elapsed time during treatment.

The internal log 56 can comprise any suitable mechanism, such as a controller in the form of a microprocessor, and can be accessed by a log input device element located on the device 12. For example, when accessed, the log information may appear on the at least one display screen 20. The internal log 56 may track, and/or store information such as the number of treatments performed, the length of each treatment, the date and time each treatment was performed, the types of tissues and/or injuries treated, and the like. In some embodiments, the internal log 56 may take the form of one or more microprocessors and/or memories.

The internal log 56 is connected to the switch 36 and the timer 38. In use, the switch 36 delivers information to the internal log 56 that then stores the received information. Additionally, the internal log 56 may store the timing information received from the timer 38. As noted above, the stored information can later be accessed via a keyboard, a touch-screen menu, through sequential depressions of the log button, and the like, and the information is displayed on the display screen 20 for analysis by the user.

The transducer cable 24 may be coupled to the transducer 14 electrically, wirelessly, optically, capacitively, inductively, by RF, by fiber optics, and the like. The transducer 14 may be a single transducer (as shown, for example, in FIG. 7), a plurality of transducers, and/or an array of transducers (as shown, for example, in FIG. 8). In addition, multiple transducers or transducer arrays may be connected or coupled through multiple cables 24 to a single device 12. Transducer arrays and multiple transducers are used when the treatment area is relatively large, and/or when there are multiple treatment areas.

In one embodiment, the one or more transducers 14 take the form of a combined frequency output transducer having multiple fixed or variable frequency transducer elements. A plurality of transducers may be used to provide concurrent therapy to various depths of thick target tissues such as inflamed joint capsules or muscle tissue.

In one exemplary embodiment, the transducer 14 may include a single transducer element or a plurality of transducer elements, and may take the form of any conventional transducer, such as those made of piezoelectric materials. In some embodiments, each transducer may comprise a generally round disc approximately 1 inch in diameter including a treatment surface 15 and a visible surface (not shown). Although described and illustrated as a generally round disc, it is understood that the transducer can have any other suitable geometric shape, and/or form.

The transducer 14 may further comprise one or more sensors 22 in the form of, for example, a temperature sensor for sensing the temperature of the patient's skin, target tissue within the body, and/or transducer treatment element during treatment. The at least one temperature sensor is electrically or otherwise connectable to the switch 36, which is configured to cease delivery of waveform information to the transducer if the temperature of the patient's skin reaches a safety threshold level, and/or predetermined value. The transducer 14 may further comprise a component physically coupled to the visible surface of the transducer 14 to alert the user to a malfunction. For example, the transducer 14 may include at least one light emitting diode (LED) 58 on the visible surface of the transducer 14, which lights up or flashes when a malfunction occurs. An LED 58 may be associated with a sensor 22 (e.g., a temperature sensor, and the like) can be placed on the control module or on both the control module 18 and the transducer treatment element. Instead of, or in addition to the LED 58, the transducer 14 may include an audible alarm 54 or vibrating element that alerts the user to the malfunction.

The device 12 may further include a powered source 30. In some embodiments, the ultrasound therapy device 12 may include a rechargeable power source in the form of at least one of a button cell, a chemical battery cell, a fuel cell, a secondary cell, a lithium ion cell, a nickel metal hydride cell, a super-capacitor, a thin film secondary cell, an ultra-capacitor, a zinc air cell, and the like. In one embodiment, for example, the ultrasound therapy device 12 includes an internal battery pack sufficient to power all elements of the device 12. In some embodiments, the device 12 may be powered by plugging it into an electrical socket.

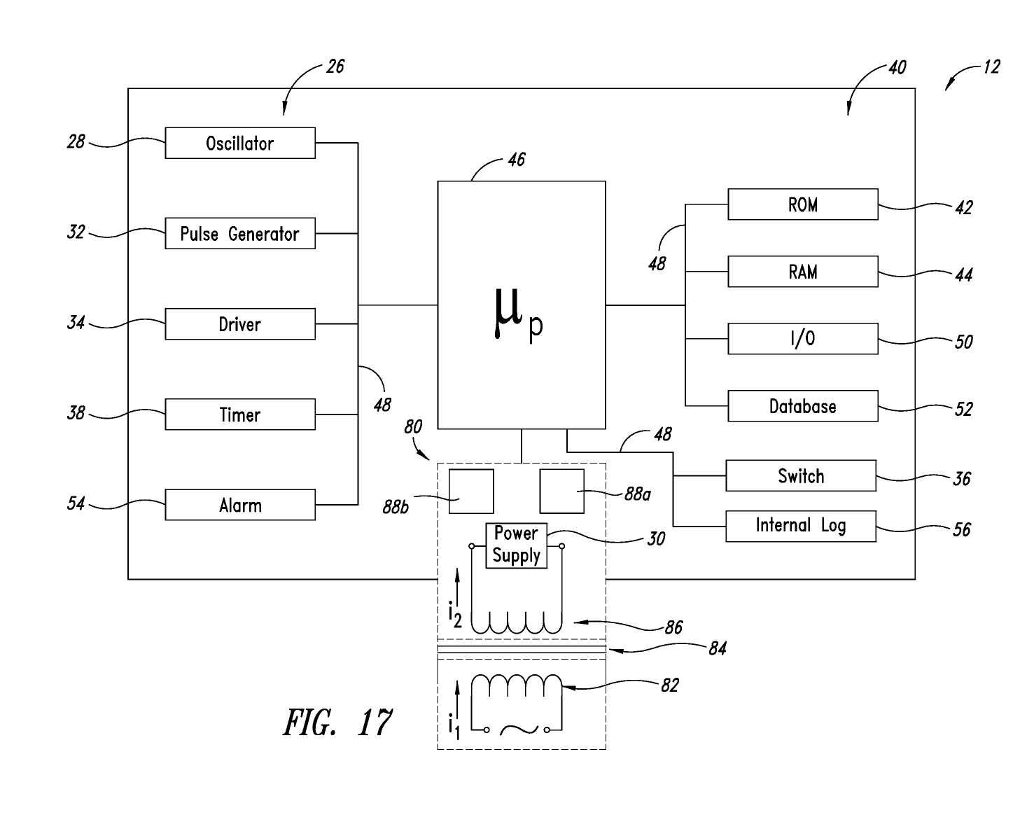

As shown in FIG. 17, the ultrasound therapy device 12 may include an inductive power supply system 80 including a primary winding 82 operable to produce a varying magnetic field 84, and a secondary winding 86 electrically or otherwise coupled to the waveform generator 26 and operable for providing a current to the waveform generator 26 in response to a varying electromagnetic field applied to the secondary winding 86. The inductive power supply system 80 may also include discrete and/or integrated circuit elements 88a, 88b to control the voltage, current and/or power delivered to the ultrasound therapy device 12.

The inductive power supply is operable to transfer energy, via inductive coupling, from one component to another through a shared magnetic field 3. A change in current flow (i₁) through one component may induce a current flow (i₂) in the other component. The transfer of energy results in part from the mutual inductance between the components. For example, the inductive power supply is operable to transfer energy, via inductive coupling, from a primary winding 82 to a secondary winding 86 through a shared magnetic field 84.

The windings 82, 86 may include one or more complete turns of a conductive material in a coil, and may comprise one or more layers. Examples of suitable conductive materials include conductive polymers, metallic materials, copper, gold, silver, copper coated with silver or tin, aluminum, and/or alloys. In some embodiments, the windings 82, 86 may comprise, for example, solid wires, including, for example, flat wires, strands, twisted strands, sheets, and the like. Examples of primary windings 82 include a coil, a winding, a primary coil, a primary winding, an inductive coil, a primary inductor, and the like. Examples of secondary windings 86 include a coil, a winding, a secondary coil, a secondary winding, an inductive coil, a secondary inductor, and the like. The secondary windings may include one or more complete turns of a conductive material in a coil, and may comprise one or more layers. The inductive power supply may be operable to provide at least one of an alternating current or a pulsed direct current to the primary winding 82. In some embodiments, the ultrasound therapy device 12 includes a rechargeable power source 88 electrically, and/or inductively coupled to the waveform generator 26, and electrically coupled in parallel with the secondary winding 86 to receive a charge thereby. In some embodiments, the rechargeable power 88 source sinks and sources voltage to maintain a steady state operation of the ultrasound device 12.

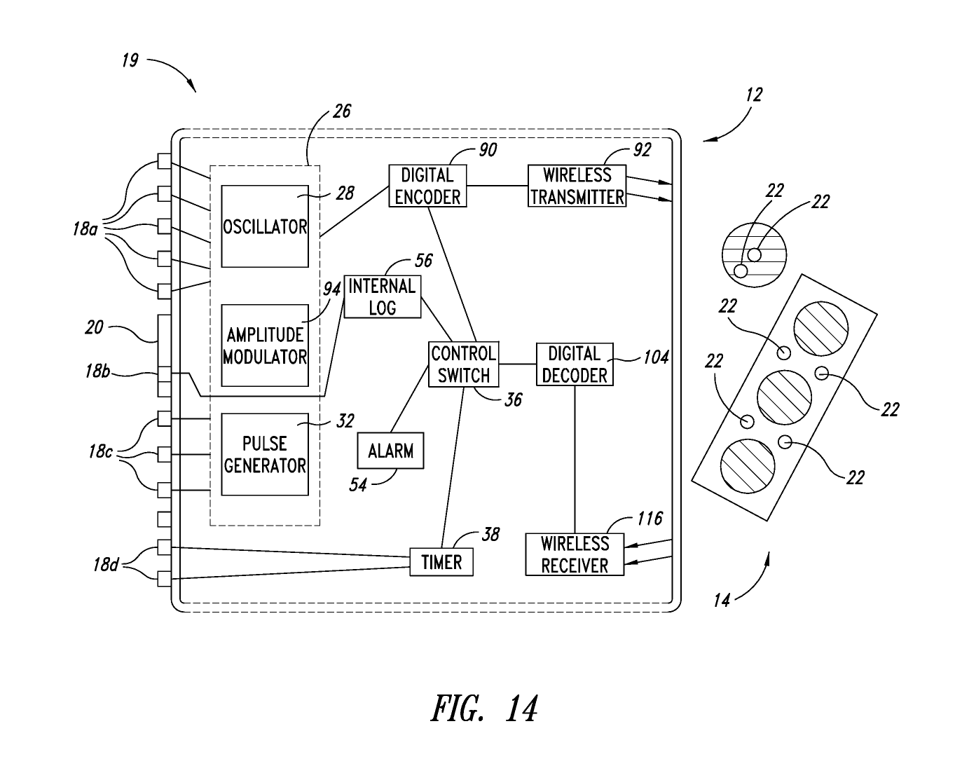

Referring to FIGS. 14, 15, and 16, the transducer 14 may be wirelessly coupled to a control module 18 that communicates with the transducer 14 via wireless communication. Examples of wireless communication include, for example, optical connections, ultraviolet connections, infrared, BLUETOOTH®, Internet connections, network connections, and the like. In some embodiments, the device 12 comprises a control module and at least one wireless transducer or transducer array. Internal batteries may allow the device 12 to be cordless and allow for maximum portability of the power the control module 18 and the transducer 14. Alternatively, the control module may remain stationary in a fixed location such that the control module 18 can be powered via an electrical socket.

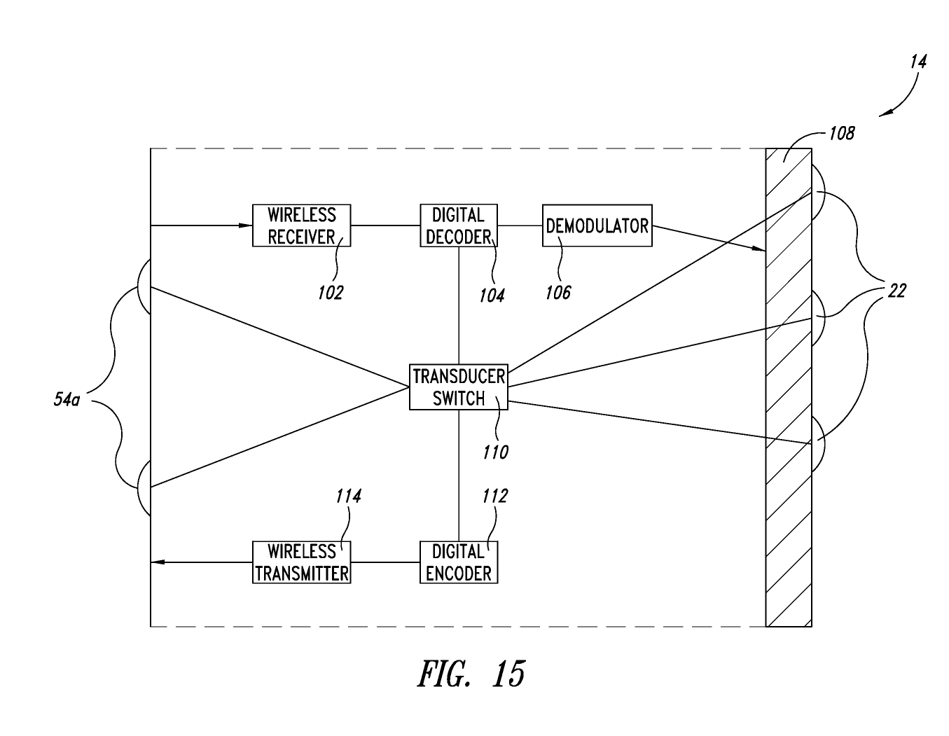

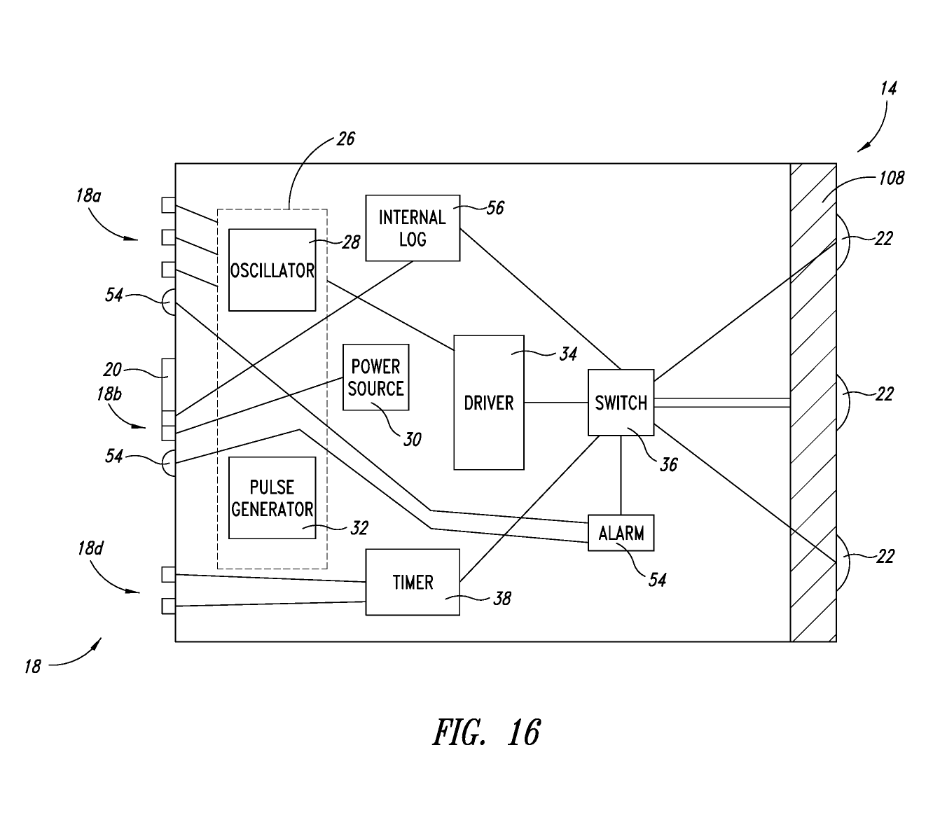

As shown in FIGS. 14 and 15, the waveform generator 26 communicates with a first digital encoder 90 which converts the signal received from the waveform generator 26 to a digital signal and delivers the digitally encoded signal to a first wireless transmitter 92. The first wireless transmitter 92 in the control module is in wireless communication with a first wireless receiver 102 in the transducer 14a. Any construction of the control module and wireless transducer 14a suitable for effecting wireless communication can be used in this embodiment.

One or more input device elements of control module 18 may communicate with a waveform generator 26. In some embodiments, the waveform generator 26 may also include an amplitude modulator 94 to modify the waveform into a form transmittable by, for example, radio waves, as is generally known. The waveform generator 26 can include a single variable frequency RF oscillator programmed to deliver different frequencies based on the type of tissue selected, or a plurality of RF oscillators, one for each type of tissue.

In one exemplary embodiment, a first wireless transmitter 92 can deliver a single wireless signal receivable by each of the first wireless receivers 102 in the one or more transducers 14a, such that each transducer 14a outputs the same waveform. Alternatively, the first wireless transmitter 92 can deliver a plurality of wireless signals, and the first wireless receivers 102 can be configured to receive a single signal unique to each individual transducer 14a such that each transducer 14a or array will output a distinct waveform. In some embodiments, the control module can include at least one input device element for inputting one or more treatment parameters associated with each of the transducer 14a. Such an embodiment, allows the physician or user to control a plurality of transducers 14a or transducer arrays from a remote location, and enables treatment of more than one patient at a time.

As shown in FIG. 15, each transducer 14 and/or array may comprise a first wireless receiver 102 for receiving wireless signals from the first wireless transmitter 92 in the control module. The first wireless receiver 102 of transducer 14 delivers the signal to a first digital decoder 104, which decodes the signal and delivers the resulting analog information to a demodulator 106. The demodulator 106 demodulates the analog and/or information and delivers the waveform to the treatment surface 108 of the transducer 14.

Each transducer 14 may also comprise at least one sensor 22 to sense the temperature of the patient's skin, target tissue within the body, and transducer treatment element during treatment and/or to sense a loss of contact between the transducer and the patient's skin. The sensor 22 communicates with a transducer switch 110, and the transducer switch 110 is configured to cease delivery of waveform information from the first digital decoder 104 to the treatment surface. The transducer may further include trouble shouting guides, alarms, indicators, and the like for alerting the user to a malfunction. For example, the transducer may include at least one light emitting diode (LED) on the visible surface of the transducer that lights up or flashes when a malfunction occurs. Alternatively, the transducer may include an audible alarm that alerts the user to the malfunction. The transducer may also include both an audible and visible alarm, such as a flashing LED coupled with an audible alarm.

The transducer switch 110 may also communicate with a second digital encoder 112. The transducer switch 110 receives information regarding the transducer 14 use and malfunctions and delivers the information to the second digital encoder 112. The second digital encoder 112 then digitally encodes the information and delivers it to a second wireless transmitter 114 in the transducer 14, which delivers the digitally encoded signal to a second wireless receiver 116 in the delivery device 12.

In some embodiments, a self-contained transducer device 12 adapted to generate and transmit a waveform for a selected tissue type may include a control module and a transducer. The self-contained transducer device 12 can be powered by an internal battery pack or super- or ultra-capacitor, allowing the self-contained transducer device 12 to be cordless, as well as maximizing device 12 portability. In some embodiments, the self-contained transducer device 12 may be designed to treat a single tissue type at a given time. The transducer device 12 may include a first input device element for selecting tissue type, a second input device elements for selecting injury type, and a third input device elements for setting the treatment time. The self-contained transducer device 12 may be operable to vary the waveform based on the type of tissue and the type of injury selected.

In some embodiments, each of the first input device elements, second input device elements, and third input device elements communicate with a waveform generator 26. In one embodiment, only one RF oscillator designed to deliver the appropriate frequency is provided. In another embodiment, the waveform generator 26 includes a plurality of RF oscillators 28, one for each tissue type. However, because the self-contained transducer device 12 is designed for maximum portability and to treat a single tissue type at a time, embodiments with only one RF oscillator 28 may be more desirable.

In some embodiments, the device 12 may include a single oscillator 28 in the form of an RF oscillator. The RF oscillator may be programmed to deliver a frequency based on the tissue type selected with the first input device element. Alternatively, the RF oscillator 28 may be programmed to deliver a single, specified frequency that is not user-selectable. In this embodiment, the need for the first input device element is eliminated.

In some embodiments, the device 12 is operable to provide therapeutic treatment of only a single tissue type. In some embodiments, a kit comprising at least two devices 12, each designed to treat a different tissue type, may be provided. For example, one kit may include at least one device 12 designed to treat bone, and at least one device 12 designed to treat tendons or ligaments. Alternatively, a kit may include a plurality of devices 12 including at least one device for treating each type of tissue.

In some embodiments, more than one waveform may be used in each treatment session. If more than one waveform is used, the user may select how the device transitions from one waveform to the next. Each waveform may be a distinct treatment point used for a selected period of time. Alternatively, the waveforms may be points on a signal wave (e.g., a sine wave, and the like) and the device may gradually sweep from one waveform to the next in discrete increments. For example, the device will gradually sweep from one waveform to the next in no less than about 5 and no more than about 10 steps. In some embodiments, the treatment time is divided generally equally between each step in the transition between waveforms. During the transition from one waveform to the next, only the pulse intensity will change until the next selected pulse intensity is achieved, at which time the remaining characteristics of the next waveform will be generated. This process continues until the treatment elapses.

The variant waveforms of the pulses and pulse series generated by the device 12 enable a generally static treatment of injuries. Previously, treatment with ultrasound required rapid and substantial movement of the treatment elements (e.g., transducers) in order to avoid thermal damage to the target and surrounding tissue. The need for constantly moving the treatment elements to maintain the thermal energy at or bellow a desire amount is reduced or substantially eliminated, in part, by varying the waveform and providing temperature sensors 22 to monitor tissue temperature during treatment. This generally static treatment of injuries significantly increases the ease of use of the ultrasound devices 12, and may significantly reduce user error, as well thermal tissue damage.

During each therapeutic treatment session, the ultrasonic treatment waveform may include multiple waveform segments, each segment having different characteristics. The total treatment time is divided between these segments as determined by the user or care provider. The user or care provider may alter the waveform or the treatment time of each segment with the first, second and third input device elements described above. Alternatively, the waveform segments can be pre-programmed into the device by the manufacturer and the user or care provider can select the pre-programmed waveform.

In some embodiments, the region of the body of the subject to be treated is first shaved and a coupling gel is applied to the skin. The transducer 14 is then placed over the coupling gel and the device 12 is used to transmit ultrasound energy to the treatment area. If the treatment area is located underneath a cast or the like, a window is cut in the cast and the coupling gel and transducer are applied to the area through the window. The first and second input device elements 12 and 14 are used to select tissue type and injury type, and treatment is commenced. During treatment, the transducer 14 is held in place by conventional techniques (e.g., compressive wraps, bandages, tape, etc.).

Many injuries have both acute and chronic components. As a result, treatment of such injuries may begin by treating the injured tissue for the acute component, and later treating the tissue for the chronic component. Alternatively, the tissue may be treated for the chronic injury first and the acute injury later. In another embodiment, both the acute and the chronic components of the injury are treated by varying the intensity, frequency, pulse intensity, duration, ratio, and repetition rate during each treatment session.

Tables 1 and 2 below list exemplary treatment parameters for each tissue type. The parameters listed in Tables 1 and 2 are for the treatment of acute injuries to the indicated tissue type with uni-variant waveform segments through which the treatment device cycles throughout the treatment time. Increases in the listed peak intensities of about 15%, and a pulse ratio of 1:3 can be used to treat sub-acute injuries to the indicated tissue type. In addition, increases in the listed peak intensities of about 25%, and a pulse ratio of 1:2 can be used to treat chronic injuries to the indicated tissue type. While a single, fixed frequency ultrasonic output wave may be used to therapeutically treat any number of tissue types, varying the frequency based upon the depth of the target tissue within the body may, for example, more accurately focus and concentrate the deposition of the ultrasound energy on the desired target tissue. This adjustment of the ultrasonic output wave's frequency based upon target tissue depth within the body may render the therapeutic ultrasound treatment more effective.

TABLE 1
TREATMENT PARAMETERS FOR ACUTE INJURIES -
UNI-VARIANT WAVEFORM SEGMENTS

| | | TENDON/ | | |
|---|---|---|---|---|
| | BONE | LIGAMENT | JOINT | MUSCLE |
| FREQUENCY | 1.5 MHz | 3 MHz | 2.5 MHz | 1 MHz |
| PULSE RATIO | 1:4 | 1:4 | 1:4 | 1:4 |
| PULSE | 1,000 Hz | 1,000 Hz | 1,000 Hz | 1,000 Hz |
| FREQUENCY | | | | |
| TOTAL | 30 min. | 35 min. | 35 min. | 30 min. |
| TREATMENT TIME | | | | |
| PULSE DURATION | 200 μsec | 200 μsec | 200 μsec | 200 μsec |

TABLE 2
PULSE INTENSITIES FOR ACUTE INJURIES -
UNI-VARIANT WAVEFORM SEGMENTS

| | | TENDON/ | | |
|---|---|---|---|---|
| | BONE | LIGAMENT | JOINT | MUSCLE |
| PHASE | begin at | begin at | begin at | begin at |
| 1 | 30 mW/cm², | 30 mW/cm², | 50 mW/cm², | 100 mW/cm², |
| | increase to | increase to | increase to | increase to |
| | 50 mW/cm² and | 50 mW/cm² | 300 mW/cm² | 500 mW/cm² |
| | decrease back | and decrease | and decrease | and decrease |
| | to 30 mW/cm² | back to | back to | back to |
| | over 5 min. | 30 mW/cm² | 50 mW/cm² | 100 mW/cm² |
| | period | over 5 min. | over 5 min. | over 5 min. |
| | | period | period | period |
| PHASE | begin at | begin at | begin at | begin at |
| 2 | 30 mW/cm², | 50 mW/cm², | 30 mW/cm², | 30 mW/cm², |
| | increase to | increase to | increase to | increase to |
| | 100 mW/cm² and | 300 mW/cm² | 100 mW/cm² | 200 mW/cm² |
| | decrease back | and decrease | and decrease | and decrease |
| | to 30 mW/cm² | back to | back to | back to |
| | over 5 min. | 50 mW/cm² | 30 mW/cm² | 30 mW/cm² |
| | period | over 5 min. | over 5 min. | over 5 min. |
| | | period | period | period |
| PHASE | begin at | begin at | begin at | begin at |
| 3 | 30 mW/cm², | 30 mW/cm², | 50 mW/cm², | 50 mW/cm², |
| | increase to | increase to | increase to | increase to |
| | 50 mW/cm² and | 100 mW/cm² | 200 mW/cm² | 300 mW/cm² |
| | decrease back | and decrease | and decrease | and decrease |
| | to 30 mW/cm² | back to | back to | back to |
| | over 5 min. | 30 mW/cm² | 50 mW/cm² | 50 mW/cm² |
| | period | over 5 min. | over 5 min. | over 5 min. |
| | | period | period | period |
| PHASE | begin at | begin at | begin at | begin at |
| 4 | 30 mW/cm², | 50 mW/cm², | 30 mW/cm², | 100 mW/cm², |
| | increase to | increase to | increase to | increase to |
| | 100 mW/cm² and | 300 mW/cm² | 100 mW/cm² | 500 mW/cm² |
| | decrease back | and decrease | and decrease | and decrease |
| | to 30 mW/cm² | back to | back to | back to |
| | over 5 min. | 50 mW/cm² | 30 mW/cm² | 100 mW/cm² |
| | period | over 5 min. | over 5 min. | over 5 min. |
| | | period | period | period |
| PHASE | begin at | begin at | begin at | begin at |
| 5 | 30 mW/cm², | 30 mW/cm², | 50 mW/cm², | 30 mW/cm², |
| | increase to | increase to | increase to | increase to |
| | 50 mW/cm² and | 50 mW/cm² | 300 mW/cm² | 200 mW/cm² |
| | decrease back | and decrease | and decrease | and decrease |
| | to 30 mW/cm² | back to | back to | back to |
| | over 5 min. | 30 mW/cm² | 50 mW/cm² | 30 mW/cm² |
| | period | over 5 min. | over 5 min. | over 5 min. |
| | | period | period | period |
| PHASE | begin at | begin at | begin at | begin at |
| 6 | 30 mW/cm², | 50 mW/cm², | 30 mW/cm², | 50 mW/cm², |
| | increase to | increase to | increase to | increase to |
| | 100 mW/cm² and | 300 mW/cm² | 100 mW/cm² | 300 mW/cm² |
| | decrease back | and decrease | and decrease | and decrease |
| | to 30 mW/cm² | back to | back to | back to |
| | over 5 min. | 50 mW/cm² | 30 mW/cm² | 50 mW/cm² |
| | period | over 5 min. | over 5 min. | over 5 min. |
| | | period | period | period |
| PHASE | — | begin at | begin at | — |
| 7 | | 30 mW/cm², | 50 mW/cm², | |
| | | increase to | increase to | |
| | | 100 mW/cm² | 200 mW/cm² | |
| | | and decrease | and decrease | |
| | | back to | back to | |
| | | 30 mW/cm² | 50 mW/cm² | |
| | | over 5 min. | over 5 min. | |
| | | period | period | |
| TOTAL | 30 min. | 35 min. | 35 min. | 30 min. |
| TREATMENT | | | | |
| TIME | | | | |

In the non-limiting examples of uni-variant waveforms listed in Tables 1 and 2, the pulse intensity is variable and begins at an initial setting (e.g., 30 mW/cm²) and increases to a peak intensity (e.g., 50 mW/cm²) in increments of no less than about 5 and no more than about 10 intensity points per treatment interval. Furthermore, as indicated by Table 2, treatment of injuries occurs in phases that may have different initial and peak intensities. As shown in Table 2, each phase is about 5 minutes in length, in which time the intensity increases from the initial intensity to the peak intensity and decreases back to the initial intensity. The treatment time may vary depending on the type of tissue being treated. In addition, although not exemplified in Tables 1 and 2, the treatment time may vary depending on the type of injury sustained by the indicated tissue as well as the chronicity of the injury being treated. It is understood that all parameters of the waveform may be user-selectable and variable. These variable waveform characteristics include intensity, frequency, pulse frequency, pulse intensity, pulse duration, pulse ratio, and pulse repetition rate. This variability in waveform characteristics enables the generally static application of ultrasonic therapy.

Tables 3 through 6 below list other exemplary treatment parameters for various tissue types. Table 3 lists exemplary parameters for the treatment of bone with multi-variant waveform segments for a total treatment time of 45 minutes. Table 4 lists exemplary parameters for the treatment of tendons and ligaments with multi-variant waveform segments for a total treatment time of 40 minutes. Table 5 lists exemplary parameter for the treatment of joints with multi-variant waveform segments for a total treatment time of 40 minutes. Table 6 lists exemplary parameters for the treatment of muscle with multi-variant waveform segments for a total treatment time of 40 minutes. Like those of Tables 1 and 2, the parameters listed in Tables 3 through 6 are for the treatment of acute injuries to the indicated tissue type with multi-variant waveform segments through which the treatment device cycles throughout the treatment time. As with the uni-variant waveforms listed in Tables 1 and 2, increases in the listed peak intensities of about 15%, and a pulse ratio of 1:3 can be used to treat sub-acute injuries to the indicated tissue type. In addition, increases in the listed peak intensities of about 25%, and a pulse ratio of 1:2 can be used to treat chronic injuries to the indicated tissue type.

TABLE 3
TREATMENT PARAMETERS FOR ACUTE INJURIES TO BONE -
MULTI-VARIANT WAVEFORM SEGMENTS

| | PHASE 1 | PHASE 2 | PHASE 3 | PHASE 4 | PHASE 5 | PHASE 6 |
|---|---|---|---|---|---|---|
| FREQUENCY | 1.5 MHz | 1.5 MHz | 1.5 MHz | 1.5 MHz | 1.5 MHz | 1.5 MHz |
| PULSE RATIO | 1:4 | 1:2 | 1:3 | 1:4 | 1:1 | 1:4 |
| PULSE | 1,000 Hz | 835 Hz | 2,500 Hz | 1,000 Hz | 5,000 Hz | 1,000 Hz |
| FREQUENCY | | | | | | |
| PULSE | 200 μsec | 400 μsec | 100 μsec | 200 μsec | 100 μsec | 200 μsec |
| DURATION | | | | | | |
| PULSE | begin at | begin at | begin at | begin at | begin at | begin at |
| INTENSITY | 30 mW/cm² | 50 mW/cm² | 30 mW/cm² | 30 mW/cm² | 30 mW/cm² | 30 mW/cm² |
| | and | and | and | and | and | and |
| | increase | increase | increase | increase | increase | increase |
| | to 50 mW/cm² | to 100 mW/cm² | to 200 mW/cm² | to 80 mW/cm² | to | to 50 mW/cm² |
| | and | and | and | and | 100 mW/cm² | and |
| | decrease | decrease | decrease | decrease | and | decrease |
| | back to | back to | back to | back to | decrease | back to |
| | 30 mW/cm² | 50 mW/cm² | 30 mW/cm² | 30 mW/cm² | back to | 30 mW/cm² |
| | | | | | 30 mW/cm² | |
| SEGMENT | 10 min | 5 min | 5 min | 10 min | 5 min | 10 min |
| TREATMENT | | | | | | |
| TIME | | | | | | |

TABLE 4
TREATMENT PARAMETERS FOR ACUTE INJURIES TO TENDONS AND LIGAMENTS -
MULTI-VARIANT WAVEFORM SEGMENTS

| | PHASE 1 | PHASE 2 | PHASE 3 | PHASE 4 | PHASE 5 | PHASE 6 |
|---|---|---|---|---|---|---|
| FREQUENCY | 3 MHz | 3 MHz | 3 MHz | 3 MHz | 3 MHz | 3 MHz |
| PULSE | 1:4 | 1:2 | 1:4 | 1:3 | 1:4 | 1:1 |
| RATIO | | | | | | |
| PULSE | 1,000 Hz | 835 Hz | 1,000 Hz | 2,500 Hz | 1,000 Hz | 5,000 Hz |
| FREQUENCY | | | | | | |
| PULSE | 200 μsec | 400 μsec | 200 μsec | 100 μsec | 200 μsec | 100 μsec |
| DURATION | | | | | | |
| PULSE | begin at | begin at | begin at | begin at | begin at | begin at |
| INTENSITY | 30 mW/cm² | 50 mW/cm² | 30 mW/cm² | 30 mW/cm² | 30 mW/cm² | 30 mW/cm² |
| | and | and | and | and | and | and |
| | increase | increase | increase | increase | increase | increase |
| | to 50 mW/cm² | to 300 mW/cm² | to 80 mW/cm² | to 200 mW/cm² | to 50 mW/cm² | to 100 mW/cm² |
| | and | and | and | and | and | and |
| | decrease | decrease | decrease | decrease | decrease | decrease |
| | back to | back to | back to | back to | back to | back to |
| | 30 mW/cm² | 50 mW/cm² | 30 mW/cm² | 30 mW/cm² | 30 mW/cm² | 30 mW/cm² |
| SEGMENT | 10 min | 5 min | 5 min | 5 min | 10 min | 5 min |
| TREATMENT | | | | | | |
| TIME | | | | | | |

TABLE 5
TREATMENT PARAMETERS FOR ACUTE INJURIES TO JOINTS -
MULTI-VARIANT WAVEFORM SEGMENTS

| | PHASE 1 | PHASE 2 | PHASE 3 | PHASE 4 | PHASE 5 | PHASE 6 |
|---|---|---|---|---|---|---|
| FREQUENCY | 2.5 MHz | 2.5 MHz | 2.5 MHz | 2.5 MHz | 2.5 MHz | 2.5 MHz |
| PULSE | 1:4 | 1:4 | 1:2 | 1:3 | 1:4 | 1:1 |
| RATIO | | | | | | |
| PULSE | 2,000 Hz | 1,000 Hz | 835 Hz | 1,250 Hz | 1,000 Hz | 5,000 Hz |
| FREQUENCY | | | | | | |
| PULSE | 100 μsec | 200 μsec | 400 μsec | 200 μsec | 200 μsec | 100 μsec |
| DURATION | | | | | | |
| PULSE | begin at | begin at | begin at | begin at | begin at | begin at |
| INTENSITY | 50 mW/cm² | 30 mW/cm² | 50 mW/cm² | 30 mW/cm² | 30 mW/cm² | 50 mW/cm² |
| | and | and | and | and | and | and |
| | increase | increase | increase | increase | increase | increase |
| | to 300 mW/cm² | to 80 mW/cm² | to 200 mW/cm² | to 100 mW/cm² | to 50 mW/cm² | to 100 mW/cm² |
| | | and | and | and | | and |
| | | decrease | decrease | decrease | | decrease |
| | | back to | back to | back to | | back to |
| | | 30 mW/cm² | 50 mW/cm² | 30 mW/cm² | | 30 mW/cm² |
| SEGMENT | 5 min | 10 min | 5 min | 5 min | 10 min | 5 min |
| TREATMENT | | | | | | |
| TIME | | | | | | |

TABLE 6
TREATMENT PARAMETERS FOR ACUTE INJURIES TO MUSCLE -
MULTI-VARIANT WAVEFORM SEGMENTS

| | PHASE 1 | PHASE 2 | PHASE 3 | PHASE 4 | PHASE 5 | PHASE 6 |
|---|---|---|---|---|---|---|
| FREQUENCY | 1 MHz | 1 MHz | 1 MHz | 1 MHz | 1 MHz | 1 MHz |
| PULSE | 1:1 | 1:4 | 1:2 | 1:4 | 1:1 | 1:4 |
| RATIO | | | | | | |
| PULSE | 1,000 Hz | 1,000 Hz | 835 Hz | 2,000 Hz | 1,665 Hz | 1,000 Hz |
| FREQUENCY | | | | | | |
| PULSE | 500 μsec | 200 μsec | 400 μsec | 100 μsec | 300 μsec | 200 μsec |
| DURATION | | | | | | |
| PULSE | begin at | begin at | begin at | begin at | begin at | begin at |
| INTENSITY | 500 mW/cm² | 30 mW/cm² | 300 mW/cm² | 30 mW/cm² | 400 mW/cm² | 30 mW/cm² |
| | and | and | and | and | and | and |
| | increase | increase | increase | increase | increase | increase |
| | to 1.5 W/cm² | to 100 mW/cm² | to 1 W/cm² | to 200 mW/cm² | to 1.2 W/cm² | to 80 mW/cm² |
| | | and | | and | and | and |
| | | decrease | | decrease | decrease | decrease |
| | | back to | | back to | back to | back to |
| | | 30 mW/cm² | | 30 mW/cm² | 400 mW/cm² | 30 mW/cm² |
| SEGMENT | 3 min | 10 min | 5 min | 7 min | 5 min | 10 min |
| TREATMENT | | | | | | |
| TIME | | | | | | |

Although principally described for treating injuries to certain tissues, device 12 can be used for any suitable purpose. For example, the devices can be used to treat joints and joint capsules. In addition, the devices can be used to promote the differentiation and/or maturation of stem cells in culture or within a human or animal body.

In some embodiments, stem cell therapy is used in conjunction with therapeutic ultrasound treatment. Often, the stem cells are first harvested from bone marrow, adipose tissue, peripheral blood from an embryo and/or an umbilical cord, or other tissue. The harvested stem cells are then implanted and/or transplanted into the injured target tissue by injecting them intralesionally, intravenously, intrathecally, intraarticularly, and the like. After injection of the stem cells, the treatment area is treated with ultrasound therapy. Upon treatment of the injected tissue with ultrasound therapy, the injured tissue, the stem cells, and the ultrasound synergistically may promote tissue healing, growth, regeneration, and repair.

According to this exemplary embodiment, the stem cells can be injected into the tissue before, after, or concurrently with the ultrasound treatment, and the ultrasound treatment would utilize pulsed or continuous waveforms having frequencies ranging from about 50 kHz to about 3 MHz and intensities ranging from about 20 mW to about 3 W. These methods of treatment (e.g., stem cell therapy used in conjunction with ultrasound treatment) can be used to treat several tissue types, including but not limited to bone, muscle, tendons, ligaments, and cartilage.

In some embodiments, a bioactive agents is used in conjunction with ultrasound therapy and stem cell therapy. The term “bioactive agent” generally refers to, without limitation, one or more compounds, molecules, or treatments that elicit a biological response from any host, animal, vertebrate, or invertebrate, including for example fish, mammals, amphibians, reptiles, birds, and humans. Examples of bioactive agents include therapeutic agents, pharmaceutical agents, pharmaceuticals (e.g., a drug, a therapeutic compound, pharmaceutical salts, and the like) non-pharmaceuticals (e.g., cosmetic substance, and the like), any of the growth factor families (e.g., insulin-like growth factors, tissue growth factors, bone growth factors, and the like), a local or general anesthetic or painkiller, an antigen or a protein or peptide such as insulin, a chemotherapy agent, an anti-tumor agent, combinations thereof, and the like

The term “bioactive agent” further refers to the active agent, as well as its pharmacologically active salts, pharmaceutically acceptable salts, prodrugs, metabolites, analogs, and the like. Non-limiting examples of suitable one or more bioactive agents include dexamethasone, TGF-beta, IGF-1, BMP-2, CDMP-2, FGF-1, all members of the bone morphogenic protein family including all cartilage-derived morphogenic proteins, all members of the tissue and transforming growth factor families, all member of the insulin-like growth factor family, all members of the fibroblast growth factor family, hyaluronans and their derivatives, and any other growth factors appropriate for assisting the differentiation and/or maturation of stem cells.

In some embodiments, at least one of the stem cells, target tissue, and body surrounding the target tissue is treated with a one or more bioactive agents. The bioactive agent assists the differentiation and/or maturation of the stem cells, and are added to the stem cells, tissue, or body either before and/or after the stem cells are injected in the target tissue and before, after or during the ultrasound therapy. Non-limiting examples of suitable one or more bioactive agents include dexamethasone, TGF-beta, IGF-1, BMP-2, CDMP-2, FGF-1, all members of the bone morphogenic protein family including all cartilage-derived morphogenic proteins, all members of the tissue and transforming growth factor families, all member of the insulin-like growth factor family, all members of the fibroblast growth factor family, hyaluronans and their derivatives, and any other growth factors appropriate for assisting the differentiation and/or maturation of stem cells.

One exemplary method for treating injured tissue includes first harvesting stem cells from bone marrow, adipose tissue, peripheral blood from an embryo, fetus, adult or an umbilical cord, or other tissue. The stem cells are treated with the one or more bioactive agents either in vitro or in vivo. The treatment area within the body may also be treated with the one or more bioactive agents before and/or after stem cell injection, and before, after or concurrently with the ultrasound treatment. The stem cells are injected into the treatment area either before and/or after being treated with the one or more bioactive agents. As noted above, the stem cells may be injected in any suitable manner, such as intralesionally, intravenously, intramuscularly, intrathecally, intraarticularly, and the like. The treatment area is then treated with ultrasound. Upon treatment of the injected tissue with ultrasound therapy, the injured tissue, the stem cells, the one or more bioactive agents and the ultrasound synergistically promote tissue healing, growth, regeneration, and repair. These methods of treatment (e.g., stem cell therapy used in conjunction with ultrasound treatment) can be used to treat several tissue types, including, but not limited to, bone, muscle, tendons and/or ligaments and cartilage.

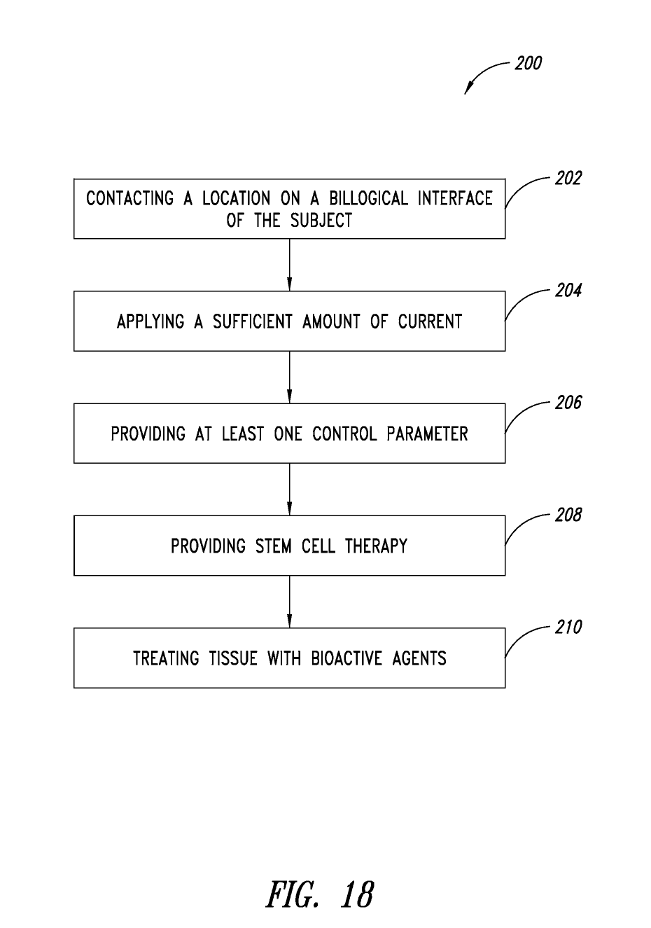

FIG. 18 shows an exemplary method 200 of treating at least one condition associated with injured tissue in a subject.

At 202, the method includes contacting a location on a biological interface of the subject with an ultrasound delivery device 12. One or more ultrasound transducers 14 are operable for providing an ultrasonic signal comprising at least a first waveform segment and a second waveform segment, the first waveform segment having at least one of an intensity, frequency, pulse intensity, pulse duration, pulse ratio, or pulse repetition rate different from the second segment.

At 204, the method further includes applying a sufficient amount of current to emit a therapeutically effective amount of ultrasonic energy from the ultrasound delivery device 12.

In some embodiments, applying a sufficient amount of current comprises applying enough current to emit a therapeutically effective amount of ultrasonic energy for at least one interval in a 24-hour period, the interval ranging from about 5 minutes to about 60 minutes. In some other embodiments, applying a sufficient amount of current comprises applying a sufficient amount current to emit a therapeutically effective amount of ultrasonic energy for at least one to three intervals in a 24-hour period, each interval independently ranging from about 5 minutes to about 60 minutes.

In some embodiments, each interval independently ranges from about 20 minutes to about 45 minutes.

At 206, the method further includes providing at least one control parameter selected from a tissue type, a treatment area, a lesion depth, a degree of injury, a treatment type, a duration type, and one or more waveform characteristics.

In some embodiments, the treatment type is selected from continuous or pulsed, and the one or more waveform characteristics are selected from an intensity, frequency, pulse intensity, pulse duration, pulse ratio, and pulse repetition rate. In some embodiments, the degree of injury is selected from an acute injury, a sub-acute injury, and a chronic injury. In some embodiments, the tissue type is selected from bone, cartilage, muscle, tendon, ligament, and stem cells, or combinations thereof.

At 208, the method further includes providing stem cell therapy before, during, and/or after emitting the therapeutically effective amount of ultrasonic energy. In some embodiments, providing stem cell therapy includes providing stem cell implantation, stem cell transplantation, stem cell delivery, and the like to the injured tissue, or combinations thereof. In some embodiments, providing stem cell therapy further includes administering one or more bioactive agents to the injured tissue.

The one or more bioactive agents may be selected from TGF-beta, IGF-1, BMP-2, CDMP-2, FGF-1, bone morphogenic proteins, cartilage-derived morphogenic proteins, tissue growth factors, transforming growth factors, insulin-like growth factors, fibroblast growth factors and hyaluronans, or combinations thereof.

At 210, the method further includes treating the injured tissue with one or more bioactive agents before, during, and/or after providing stem cell therapy to the injured tissue, and treating the injured tissue with one or more bioactive agents before, during, and/or after emitting the therapeutically effective amount of ultrasonic energy.

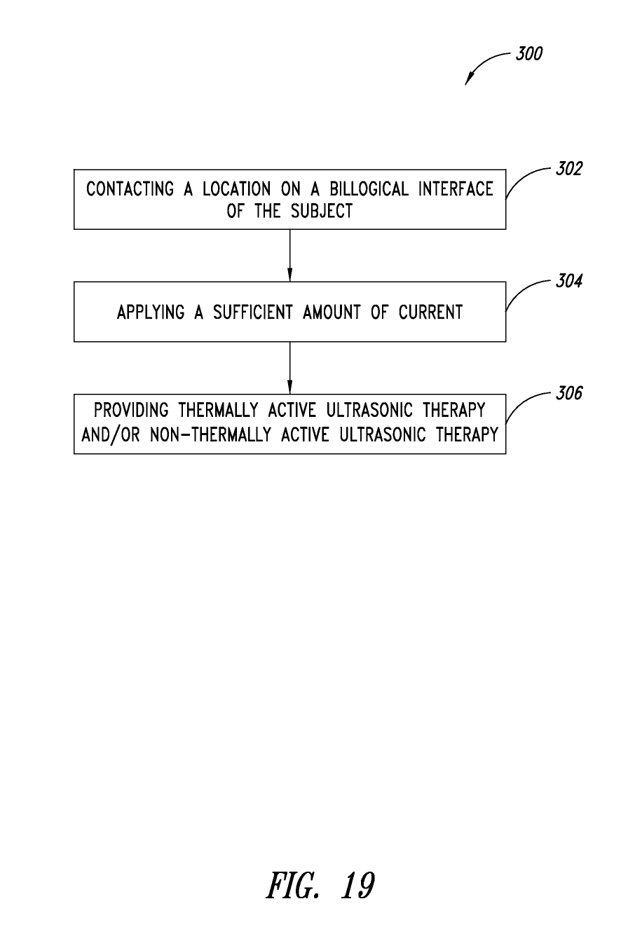

FIG. 19 shows an exemplary method 300 of providing thermal and non-thermal ultrasonic treatment to a subject.

At 302, the method includes contacting a location on a biological interface of the subject with an ultrasound delivery device 10, the ultrasound delivery device 10 including one or more ultrasound transducers 14. In some embodiments, the one or more ultrasound transducers 14 are configured to provide thermally active moderate-intensity ultrasonic energy, and non-thermally active low-intensity ultrasonic energy.

At 304, the method includes applying a sufficient amount of current to the one or more ultrasound transducers 14 to emit a therapeutically effective amount of the thermally active moderate-intensity ultrasonic energy and the non-thermally active low-intensity ultrasonic energy from the ultrasound delivery device.

In some embodiments, the one or more ultrasound transducers 14 may include a plurality of piezoelectric crystals 14d, 14e, and applying a sufficient amount of current to the one or more ultrasound transducers 14 may include delivering a pulsed signal having a first waveform to a first plurality of the plurality of piezoelectric crystals 14d, 14e, and delivering a pulsed signal having a second waveform to a second plurality of the plurality of piezoelectric crystals 14d, 14e spatially adjacent to the first plurality; such that no two spatially adjacent piezoelectric crystals 14d, 14e within the first or the second pluralities emit the same thermal or non-thermal waveforms.

In some embodiments, applying a sufficient amount of current to the one or more ultrasound transducers 14 includes applying a sufficient amount of current to concurrently or sequentially emit a therapeutically effective amount of a thermally active ultrasonic waveform having a spatial average-temporal average intensity in the range of about 0.25 watts per square centimeter to about 3 watts per square centimeter, and a non-thermally active ultrasonic waveform having a spatial average intensity in the range of about 0.01 watts per square centimeter to about 0.25 watts per square centimeter.

In some embodiments, applying a sufficient amount of current to the one or more ultrasound transducers 14 includes applying a sufficient amount of current to concurrently emit from the one or more the one or more ultrasound transducers a therapeutically effective amount of a thermally active ultrasonic waveform having a spatial average-temporal average intensity in the range of about 0.25 watts per square centimeter to about 3 watts per square centimeter, and a non-thermally active ultrasonic waveform having a spatial average-temporal average intensity in the range of about 0.01 watts per square centimeter to about 0.25 watts per square centimeter.

In some embodiments, applying a sufficient amount of current to the one or more ultrasound transducers may comprise applying a sufficient amount of current to each of the one or more transducer based upon measurements inherent to each transducer or as called for in a transducer identification circuit.

The method may further include emitting the thermally active ultrasonic waveform in a continuous fashion for a period of time ranging from about 7 minutes to about 10 minute. In some embodiments, the method may further include emitting the non-thermally active ultrasonic waveform in a pulsed fashion for a period of time ranging from about 7 minutes to about 10 minute.

At 306, the method may further include alternating between providing the therapeutically effective amount of the thermally active moderate-intensity ultrasonic energy and the non-thermally active low-intensity ultrasonic energy to the subject. In some embodiments, the method may include alternating between providing the therapeutically effective amount of the thermally active moderate-intensity ultrasonic energy and the non-thermally active low-intensity ultrasonic energy to the subject. In some embodiments, the method may include concurrently providing the therapeutically effective amount of the thermally active moderate-intensity ultrasonic energy and the non-thermally active low-intensity ultrasonic energy to the subject.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

As one skill in the relevant art would readily appreciate, the present disclosure comprises methods of treating a subject by any of the compositions and/or methods described herein.

Aspects of the various embodiments can be modified, if necessary, to employ systems, circuits, and concepts of the various patents, applications, and publications to provide yet further embodiments, including those patents and applications identified herein. While some embodiments may include all of the waveform generators, treatment control modules, RF generators, and other structures discussed above, other embodiments may omit some of the waveform generators, treatment control modules, RF generators, and other structures. Still other embodiments may employ additional ones of the waveform generators, treatment control modules, RF generators, and other structures generally described above. Even further embodiments may omit some of the waveform generators, treatment control modules, RF generators, and structures described above while employing additional ones of the waveform generators, treatment control modules, RF generators, and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

What is claimed is:

1. An ultrasound therapy device for delivering ultrasonic treatment to a biological entity, comprising:
at least one waveform generator configured to generate a first set of drive signals and at least a second set of drive signals, the first set of drive signals and the second set of drive signals having an average frequency selected from a range of about 50 kHz to about 4 MHz;
one or more transducers drivingly coupled to the waveform generator, to receive the first set of drive signals and the second set of drive signals and to concurrently generate a first ultrasonic signal and a second ultrasonic signal based on the first set of drive signals and the second set of drive signals, the first ultrasonic signal having a spatial average-temporal average intensity in a range of about 0.25 watts per square centimeter to about 3 watts per square centimeter, and the second ultrasonic signal having a spatial average-temporal average intensity in a range of about 0.01 watts per square centimeter to about 0.25 watts per square centimeter; and
a programmable controller communicatively coupled to the one or more transducers, the programmable controller operable to control the first set of drive signals and the second set of drive signals to the one or more transducers such that the one or more transducers operate in a pre-selected sequence, for a pre-selected period of time.

2. The ultrasound therapy device of claim 1 wherein the first ultrasonic signal is operable to provide thermal ultrasonic therapy to the biological entity and the second ultrasonic signal is operable to provide non-thermal ultrasonic therapy to the biological entity.

3. The ultrasound therapy device of claim 2 wherein the one or more transducers include at least two spaced apart piezoelectric crystals each operatively coupled to the programmable controller and configured to concurrently receive the first set of drive signals and the second set of drive signals and deliver a constant or pulsed thermal ultrasonic therapy and a constant or pulsed non-thermal ultrasonic therapy to the biological entity.

4. The ultrasound therapy device of claim 1 wherein the first set of drive signals comprises a first drive signal train having at least a first waveform segment and a second waveform segment different from the first waveform segment; and the second set of drive signals comprises a second drive signal train having at least a third waveform segment and a fourth waveform segment different from the third waveform segment.

5. The ultrasound therapy device of claim 4 wherein the second waveform segment has at least one of an intensity, a frequency, a pulse intensity, a pulse duration, a pulse frequency, a pulse ratio, or a pulse repetition rate different from the first waveform segment; and wherein the fourth waveform segment has at least one of an intensity, a frequency, a pulse intensity, a pulse duration, a pulse frequency, a pulse ratio, or a pulse repetition rate different from the third waveform segment.

6. The ultrasound therapy device of claim 1, further comprising a coupling element for removably-attaching the one or more transducers to the biological entity, the coupling element configured to maintain the one or more transducers substantially stationary during delivery of the ultrasonic treatment.

7. The ultrasound therapy device of claim 1 wherein the one or more transducers are configured to receive the first set of drive signals and the second set of drive signals and to generate a first non-thermal ultrasonic waveform and a second thermal ultrasonic waveform based on the first set of drive signals and the second set of drive signals.

8. The ultrasound therapy device of claim 1 wherein the one or more transducers are configured to receive the first set of drive signals and the second set of drive signals and to concurrently generate a first non-thermal ultrasonic waveform and a second thermal ultrasonic waveform based on the first set of drive signals and the second set of drive signals.

9. The ultrasound therapy device of claim 1 wherein at least one of the one or more transducers includes two or more spatially adjacent piezoelectric crystals each piezoelectric crystal configured to generate either a thermal waveform or a non-thermal waveform.

10. The ultrasound therapy device of claim 1 wherein at least one drive signal of the first set comprises a continuous digital signal, and wherein the at least one drive signal of the second set comprises a pulsed digital signal.

11. A method for providing thermal and non-thermal ultrasonic treatment to a subject, comprising
contacting a location on a biological interface of the subject with an ultrasound delivery device, the ultrasound delivery device comprising one or more ultrasound transducers, the one or more ultrasound transducers configured to provide thermally active moderate-intensity ultrasonic energy, and non-thermally active low-intensity ultrasonic energy; and
applying a sufficient amount of current to the one or more ultrasound transducers to emit a therapeutically effective amount of the thermally active moderate-intensity ultrasonic energy and the non-thermally active low-intensity ultrasonic energy from the ultrasound delivery device concurrently.

12. The method of claim 11 wherein applying the sufficient amount of current to the one or more ultrasound transducers comprises concurrently emitting a therapeutically effective amount of a thermally active ultrasonic waveform having a spatial average-temporal average intensity in a range of about 0.25 watts per square centimeter to about 3 watts per square centimeter, and a non-thermally active ultrasonic waveform having a spatial average-temporal average intensity in a range of about 0.01 watts per square centimeter to about 0.25 watts per square centimeter.

13. The method of claim 11 wherein applying the sufficient amount of current to the one or more ultrasound transducers comprises concurrently emitting from the one or more ultrasound transducers a therapeutically effective amount of a thermally active ultrasonic waveform having a spatial average-temporal average intensity in a range of about 0.25 watts per square centimeter to about 3 watts per square centimeter, and a non-thermally active ultrasonic waveform having a spatial average-temporal average intensity in a range of about 0.01 watts per square centimeter to about 0.25 watts per square centimeter.

14. The method of claim 11 wherein applying the sufficient amount of current to the one or more ultrasound transducers comprises sequentially emitting from the one or more ultrasound transducers a therapeutically effective amount of a thermally active ultrasonic waveform having a spatial average-temporal average intensity in a range of about 0.25 watts per square centimeter to about 3 watts per square centimeter, and a non-thermally active ultrasonic waveform having a spatial average-temporal average intensity in a range of about 0.01 watts per square centimeter to about 0.25 watts per square centimeter.

15. The method of claim 11, further comprising applying current to each of the one or more transducer based upon measurements inherent to each transducer or provided by a transducer identification circuit.

16. The method of claim 11, further comprising:
emitting the thermally active ultrasonic waveform in a continuous fashion for a period of time ranging from about 3 minutes to about 10 minutes; and
emitting the non-thermally active ultrasonic waveform in a pulsed fashion for a period of time ranging from about 5 minutes to about 60 minutes.

17. The method of claim 11 wherein the one or more ultrasound transducers include a plurality of piezoelectric crystals, and wherein applying the sufficient amount of current to the one or more ultrasound transducers comprises delivering a pulsed signal having a first waveform to a first plurality of the plurality of piezoelectric crystals; and delivering a pulsed signal having a second waveform to a second plurality of the plurality of piezoelectric crystals spatially adjacent to the first plurality such that no two spatially adjacent piezoelectric crystals within the first or the second pluralities of piezoelectric crystals emit the same thermal or non-thermal waveforms.

18. The method of claim 11, further comprising:
alternatingly providing the thermally active moderate-intensity ultrasonic energy and the non-thermally active low-intensity ultrasonic energy to the subject.

19. An ultrasound therapy system for delivering thermally active moderate-intensity waveforms and non-thermally active low-intensity waveforms, the system comprising:
an ultrasound delivery device including a first waveform generator configured to generate a first drive signal, at least a second waveform generator configured to generate a second drive signal, and one or more transducers communicatively coupled to the first and second waveform generators, the one or more transducers configured to receive the first drive signal and the second drive signal and to concurrently generate a first ultrasonic signal and a second ultrasonic signal based on the first drive signal and the second drive signal, the first ultrasonic signal having a spatial average-temporal average intensity in a range of about 0.25 watts per square centimeter to about 3 watts per square centimeter, and the second ultrasonic signal having a spatial average-temporal average intensity in a range of about 0.01 watts per square centimeter to about 0.25 watts per square centimeter; and
a controller configured to communicate at least one instruction to the first and the second waveform generators, and to the one or more transducers.

20. The system of claim 19 wherein the at least one instruction includes at least one of an encrypted data stream, an activation code, an authorization instruction, an authentication data stream, a prescription ultrasonic dosing instruction, and an ultrasound dose administration instruction according to a prescribed regimen.

21. The system of claim 19 wherein the controller wirelessly communicates the at lest one instruction via an optical connection, an ultraviolet connection, an infrared connection, a BLUETOOTH® connection, an Internet connection, or a network connection.

22. The system of claim 19, further comprising:
a inductive power supply including a primary winding operable to produce a varying magnetic field; and
a secondary winding electrically coupled to at least one of the first and second waveform generators and operable for providing a potential to the at least one of the first and second waveform generators in response to a varying electromagnetic field applied to the secondary winding; and one or more transducers electrically coupled to the at least one of the first and second waveform generators, the one or more transducers configured to receive the first drive signal and to generate a first ultrasonic signal based in part on the first drive signal.

23. The system of claim 22 wherein the ultrasound delivery device is physically distinct from the inductive power supply.

24. The system of claim 22 wherein the inductive power supply is operable to provide at least one of an alternating current or a pulsed direct current to the primary winding.

25. The system of claim 22 wherein the ultrasound therapy device includes a rechargeable power source electrically coupled to at least one of the waveform generators, and electrically coupled in parallel with the secondary winding to receive a charge thereby.

26. The system of claim 22, further comprising a controller including an intensity modifying circuit operable to dampen or boost at least one of the first drive signal and the second drive signal such that some of the one or more transducers produce waveforms of only moderate intensity or low-intensity.

27. A method for providing thermal and non-thermal ultrasonic treatment to a subject, comprising:
generating a first set of drive signals and at least a second set of drive signals, the first set of drive signals and the second set of drive signals having an average frequency selected from a range of greater than about 50 kHz to less than about 4 MHz; and
concurrently generating a first ultrasonic signal and a second ultrasonic signal based on the generated first set of drive signals and the second set of drive signals, the first ultrasonic signal operable to provide a therapeutically effective amount of thermally active moderate-intensity ultrasonic energy to the subject and the second ultrasonic signal operable to provide non-thermally active low-intensity ultrasonic energy to the subject.
